(19) United States
(12) Patent Application Publication
(10) Pub. No.: US 2017/0009255 A1
(43) Pub. Date: Jan. 12, 2017

(54) METHOD FOR PRODUCING A FUSION MIXTURE FOR TRANSFER OF A CHARGED MOLECULE INTO AND/OR THROUGH A LIPID MEMBRANE

(71) Applicants: beniag GmbH, Julich (DE); ibidi GmbH, Martinsried (DE)

(72) Inventors: Bernd Hoffmann, Julich (DE); Agnes Csiszar, Julich (DE); Nils Hersch, Wegberg (DE); Rudolf Merkel, Julich (DE); Marco Hoffmann, Wegberg (DE); Roman ZANTL, Baldham (DE)

(21) Appl. No.: 15/205,967

(22) Filed: Jul. 8, 2016

(30) Foreign Application Priority Data
Jul. 9, 2015 (EP) 15176005.5

(51) Int. Cl. C12N 15/88; B01J 13/10

(52) U.S. Cl. CPC C12N 15/88; B01J 13/10

(57) ABSTRACT

A method for producing a fusion mixture for a transfer of a charged molecule into and/or through a lipid membrane is disclosed. In an embodiment, the method comprises: providing an initial mixture comprising a positively charged amphipathic molecule A, an aromatic molecule B with hydrophobic range and a neutral, amphipathic molecule C, whereby the molecule types are at hand in a ratio A:B:C of 1-2:0.02-1:0-1 mol/mol; generating a fusogenic liposome by absorption of the initial mixture in a watery solvent; providing a charged molecule; forming a complex from the charged molecule and a neutralizing agent; and incubating the complex with the fusogenic liposome so that a fusion mixture is obtained.

### CROSS-REFERENCE TO RELATED APPLICATION

This application claims the benefit of priority to European patent application No. 15176005.5, filed Jul. 9, 2015, which is incorporated herein by reference in its entirety as though fully set forth herein.

### TECHNICAL FIELD

The invention relates to a method for producing a fusion mixture for a transfer of a charged molecule into and/or through a lipid membrane as well as to a method for transfer of a charged molecule into and/or through a lipid membrane by means of a fusion.

### BACKGROUND

For most diverse applications, e.g. in the field of basic research, biotechnology or therapy, as well, there is a need for systems, by means of which charged molecules, as e.g. DNA or different RNA molecules are transmitted into living cells.

Two non-viral main transfer mechanisms for charged molecules are known and are routinely applied. For the case of nucleic acids, these are transfers by means of endocytosis (lipofection) as well as by means of electroporation.

From Khalil et al. (I:A. Khalil, K. Kogure, H. Akita, H. Haraschima, 2006. Uptake Pathways and Subsequent Intracellular Trafficking in Nonviral Gene Delivery. PHARMACOLOGICAL REVIEWS, Vol. 58, No. 1, 32-45) it is known to differentiate between endocytotic and non-endocytotic absorption processes for DNA. In the economic community, it is accepted that the main way for the absorption of DNA by means of lipoplexes (also called cationic lipid DNA complexes) under standard conditions occurs by the endocytotic manner. As endocytosis, the vesicular absorption of extracellular macromolecules is designated, which is thereafter enzymatically degraded in lysosomes and is applied for own metabolic processes. Through this, generally, the functional characteristics of the absorbed macromolecules get completely lost. Therefore, the direct absorption of macromolecules, as postulated until the mid-90ies, after adhesion of the lipids of the lipoplexes with the slightly negatively charged cell membrane is not sustainable, but at best involved with a very small proportion.

This is also proven by the work of Friend et al., according to which DNA in intracellular inclusions via electron microscope photographs was shown, which only could be the result from endocytosis (Friend D S, Papahadjopoulos D, and Debs R J (1996) Endocytosis and intracellular processing accompanying transfection mediated by cationic liposomes. Biochim Biophys Acta 1278:41-50). This form of recording is also confirmed by Weijun und Szoka Jr. (Weijun Li and Francis C. Szoka Jr. 2007. Lipid-based Nanoparticles for Nucleic Acid Delivery. Pharmaceutical Research, 24, 438-449.).

An alternative to the endocytotic absorption mechanism is the electroporation. Thereby, as regarding the endosomal absorption mechanism, in a first step lipoplexes are generated, which due to their charge build up a first fusion to the cell surface. Afterwards, by applying a short-time applied voltage pulse, the cell membrane is destabilized in a manner that by means of temporary pore formation, lipoplexes reach the inside of the cell, the so-called cytoplasm of the cell. Electroporation uses the phenomenon of the destabilization of lipid membranes, if their natural electrical resistance by appliance of an electrical pulse in the nanosecond to microsecond range is neutralized (Tsong, Biophysical Journal, 1991 60:297-306, Electroporation of cell membranes; Weaver, Journal of Cell Biochemistry, 1993, 51:426-435, Electroporation: a general phenomenon for manipulating cells and tissues). Formed pores in the cell membrane are maintained for some milliseconds and allow by means of passive diffusion a rapid molecule exchange between the inside of the cell and the surrounding medium.

For the development of the lipoplexes necessary for the endosomal as well as the non-endosomal transfer mechanism, always cationic lipids and/or in interaction positively lipids charged with multivalent cations are used, which due to charge interactions with negatively charged nucleic acids build up high molecular lipoplexes. Such lipoplexes can build up most different conformations and/or lipidic phases (Tresset, PMC Biophysics, 2009, 2:3, The multiple faces of self-assembled lipidic systems), whereby the proportion of positively charged lipids is always high. As the cationic lipids almost do not occur in biological systems, after insertion into living systems, they induce a number of toxic effects up to lethality, which increase with rising quantity of the respective lipids (Dass, Journal of Molecular Medicine, 2004, 82:579-591, Lipoplex-mediated delivery of nucleic acids: factors affecting in vivo transfection).

It is known that the development of cationic liposomes from the initial components is difficult to control, as different structures from the initial substances may be developed. For the development of liposomes, cationic lipids therefore are preliminarily regularly mixed with neutral lipids as so-called aiding lipids, as cationic lipids alone obviously cannot guarantee the liposome development. As aide e.g. DOPE or cholesterol are used, as also known from the above mentioned literature Khalil et al.

As a further method for the transfer of molecules, the fusion of fusogenic liposomes with lipid membranes is known. Based on identical lipid types, as described above for positively charged as well as neutral lipids, such fusogenic liposomes have at least a further third component, which is an aromatic lipid (Csiszár A. et al. Novel Fusogenic Liposomes for Fluorescent Cell Labeling and Membrane Modification, Bioconjugate Chemistry, 2010, 21, 537-543; Kleusch Ch. et al. Fluorescent lipids: functional parts of fusogenic liposomes and tools for cell membrane labeling and visualization, Molecules, 2011, 16, 221-250). Here, a fusion of the liposomes occurs with a further lipid membrane, e.g. a cell membrane in a ratio of 1:0.1:1 (positive lipid:aromatic component:neutral lipid). Possible mixing ratios for a fusion are also described in WO 2011/003406 and WO 2014/154203.

A transfer of charged molecules by means of fusogenic liposomes is difficult and insufficiently efficient, as the charged molecules reduce the charge density of the positive lipids (partial neutralization). As fusogenic liposomes mainly are simple lipid-coated systems, to which or into which molecules of interest are introduced, the concentration of cationic lipids is clearly lower than regarding classical lipoplex transfer mechanisms. Additionally, such lipids are only infiltrated into the plasm membrane and are not also directly transported into the lumen of the cell.

As electrostatic interactions of the transfer system with the target membrane are of great importance for all above described techniques, explicitly for the transfer of charged molecules, in particular nucleic acids, the attempt had been made to reduce the strong negative charge by using DNA neutralizing agents. By means of different molecules, neutralization is possible. Binding proteins, protein fragments or peptides with a large number of alkaline amino acids (Wienhues et al., DNA, 1987, 6:81-89, A novel method for transfection and expression of reconstituted DNA-protein complexes in eukaryotic cells) had been used for this purpose as well as positively charged polymers (Boussif et al. PNAS, 1995, 92:7297-7301, A versatile vector for gene and oligonucleotide transfer into cells in culture and in vivo: polyethylenimine) or even divalent ions (Haberland et al. Biochim, Biophys Acta, 1999, 14:21-30, Calcium ions as efficient cofactor of polycation-mediated gene transfer). For complexes from DNA and proteins/peptides, as e.g. polylysine, protamine or adenoviral central proteins, it could be shown that these effect a direct improvement of the absorption efficiency of DNA in living cells even without additional use of liposomal systems (Wienhues et al., see above). Thereby, DNA and proteins/peptides form homogenous, densely packed nanoparticles (polyplexes, also nanoplexes), which through this are better protected by enzymatic degradation. These nanoparticles furthermore can be introduced into cells by means of endosomal transfection, as well as electroporation in form of lipopolyplexes in order to there increase the transfection efficiency (Chen et al. Biomaterials, 2011, 32:1412-1418, Transfection efficiency and intracellular fate of polycation liposomes combined with protamine). The increase of the transfection efficiency is thereby probably caused by a protamine mediated and improved discharge from lysosomes as well as by transport into the cell core.

Similar results as for cationic proteins/peptides are also described for polycationic polymers, as e.g. polyethylenimine (PEI) (Boussif et al. PNAS, 1995, 92:7297-7301, A versatile vector for gene and oligonucleotide transfer into cells in culture and in vivo: polyethylenimine). Therefore, also here polyplexes can be generated in the presence of nucleic acids and can be either directly or in combination with liposomes infiltrated into cells in order to increase there the functional introduction of nucleic acids by means of reduced enzymatic degradation of the DNA, improved release of lysosomes as well as better transport of the DNA into the cell core.

Divalent ions typically serve during transfer of charged molecules as supporting units. In combination, especially with endocytotic absorption mechanisms, divalent ions, in particular Ca²⁺, presumably build up microprecipitates (calcium phosphate for Ca²⁺). Such microprecipitates have membranolytic activity and presumably effect thereby an improved release of nucleic acids from lysosomes by destroying the same (Haberland et al. Biochim, Biophys Acta, 1999, 14:21-30, Calcium ions as efficient cofactor of polycation-mediated gene transfer).

Disadvantageous for the transfer of nucleic acids by means of endosomal absorption (lipofection) is the degradation mechanism of nucleic acids naturally occurring in endosomes inside the cells. By means of artificial mechanisms, thus, endosomes need to be hindered in their functionality and have to be caused to resolve and to release absorbed substances. This intervention into the endosomal functionality mechanism constitutes a significant intervention and, thus, stress for the living cells, whereby natural functionalities may be changed. The stress level is furthermore increased by the fact that the lipoplexes generated for the transfer of nucleic acids have a high proportion of positively charged lipids, which are rather build up by cells themselves and therefore may lead to lethality, but even with reduced concentration may significantly damage the microdomain structure of biological membranes (Dass, J. Pharm Pharmacol, 2002, 54:593-601, Cytotoxicity issues pertinent to lipoplex-mediated gene therapy in-vivo). In addition, different cell types are characterized by a strongly varying endocytosis activity, whereby the efficiency of lipofection may be strongly affected.

In case of electroporation, the stress levels caused by the method are so high that up to 90% of the applied cells do not survive the procedure (Vernhes et al. Bioelectrochem Bioenerg. 1999, 48:17-25, Chinese hamster ovary cells sensitivity to localized electrical stresses). This disadvantage causally takes place by the generating nanopores of different sizes in the outer cell membrane due to which the natural and most accurately regulated ions establishment of the cell is severely disturbed. Such stress levels effect for the case of electroporation as well as for lipofection inhomogeneous transfer quantities of lipoplexes, whereby also afterwards, the analysis of the function to be examined is subject to an increased heterogeneity.

While high stress values at target cells and low efficiencies during the liposomal fusion are clearly reduced, this method has the disadvantage to react very sensitively to changes of the electrostatic interaction between fusogenic liposome as well as target membrane. Already small quantities of charged molecules thereby change the interaction between liposomes and target membrane in such a manner that after coupling of the liposomes, a following fusion of both membranes is partially to completely impeded and through this, also fusogenic liposomes are only absorbed endocytotically with the disadvantages connected therewith. As mixtures from cationic lipids with polyanions, as e.g. DNA or RNA as described above, tend to complexing in lipoplexes, in case of fusion impedance also such a complex generation with high transfer of positively charged lipids is probable as by means of electroporation and/or lipofection.

### BRIEF SUMMARY

The objective technical problem of the invention therefore is to provide an improved fusion mixture for transfer of a charged molecule. This problem is solved by the subject matter of claim 1.

According to the invention, a method for producing a fusion mixture for a transfer of a charged molecule in and/or through a lipid membrane is provided including the steps:
a) providing an initial mixture comprising a positively charged amphipathic molecule A, an aromatic molecule B with hydrophobic range (region) and a neutral, amphipathic molecule C, whereby the molecule types are at hand in a ratio A:B:C of 1-2:0.02-1:0-1 mol/mol,
b) generating a fusogenic liposome by absorption (reception) of the initial mixture in a watery solvent,
c) providing a charged molecule,
d) forming a complex from the charged molecule and a neutralizing agent,
e) incubating the complex with the fusogenic liposome so that a fusion mixture is obtained.

Surprisingly, it emerged that this method leads to a mixture which allows in a reliable manner a transfer by means of fusion into and/or through a target membrane in the form of a lipid membrane, during which the transferred molecules maintain their functionality and the entire process is accompanied with low stress values for the target membrane. This sequence of steps (in the sequence according to the claim) leads to an advantageous embedding of the complex into the fusogenic liposome. The arising fusogenic complex liposome is not a classic lipoplex, which would be absorbed by means of endocytosis.

Surprisingly, in particular the sequence of steps according to the claim leads to the desired excellent fusion characteristics. Starting from the prior art regarding the transport of electrically neutral molecules e.g. in cells, also the person skilled in the art would actually consider that it would be the most effective manner if the product from step e) would be taken for the watery solvent used in step b). This would mean that initially, a solvent is generated from charged molecule and neutralized agent, with which then the initial mixture is mixed (and soaked) in order to produce the fusogenic liposome. Therefore, this approach is more obvious, as it is expected that the complex from charged molecule and neutralized agent is absorbed with a higher probability into the lumen of the newly formed liposome, if the complex is present in the solvent, which is used for soaking. The particle transport, however, surprisingly works essentially more effectively, if the complexes are initially given to the liposomes after the same had been formed.

The target membrane/lipid membrane may be a biological membrane (or a component thereof) or an artificially produced lipid membrane. Examples for a lipid membrane are a target membrane including plasm or cell organelle membranes, a component of a cell membrane, a purified biological membrane or even an artificial membrane system.

The charged molecule may be dissociated (thus, e.g. protonated) or may be present in ionic form. The charged molecule may in particular be a protein, a peptide, an amino acid, a polynucleotide, a nucleic acid, a poly-, oligo- or disaccharide, an antibiotic, an antiseptic, a cytostatic, an immunosuppressive drug, a therapeutically relevant polymer (e.g. polymaleic acid, hyaluronic acid, functionalized dextran, hydrogels), a dendrimer, a chelator, a surface functionalized nano- or microparticle (e.g. polystyrene, ferritin), a microswimmer and/or a dye. This charged molecule may be of natural origin, as e.g. from extracted from cells and where appropriate purified proteins or polynucleotides, or a synthetically produced molecule as e.g. multiply charged polypeptides or polynucleotides.

In step a), the molecules of the initial mixture may already be available in a watery solution or may be transferred as dry lipid mixture and/or from an organic solvent after drying into a watery solution. The watery solution, thus, constitutes a liposome buffer. This buffer preferably has an osmolarity of less than 100 mOsm. Its pH-value may be in the range of 7.0 to 8.0. The watery solution may e.g. be HEPES, TRIS, HEPPS or also a phosphate buffer.

Step d) may occur in a manner that the complex has a zeta-potential of −50 mV to 0 mV, in particular of −50 mV to −10 mV. This leads to an improved reliability of the transfer of charged molecules by means of fusion and clearly reduces the probability of an absorption by means of endocytosis. In the following, the complex from charged molecule and neutralizing agent is sometimes also called complex A.

The neutralizing agent in particular has a positive or negative charge. The charge of the neutralizing agent has a sign opposite to the charge of the charged molecule. In case of negatively charged molecules (e.g. DNA, RNA or siRNA), thereby a positively charged agent, in case of positively charged molecules (e.g. alkaline peptides/proteins (histones, avidin) or cationic polymers), a negatively charged agent is used.

The neutralizing agent for a negatively charged molecule leads to the fact that the entire charge of the complex compared to the charge of the charged molecule is switched to the direction of a neutral charge. The neutralizing agent for a positively charged molecule leads to the fact that the entire charge of the complex compared to the charge of the charged molecule is switched beyond a neutral charge into the anionic range. Independent of the sign of the charge of the charged molecule, the indicated range of the zeta potential emerged to be explicitly advantageous.

In case of negatively charged molecules, for the neutralization/charge switching in particular a polycationic polymer or an alkaline (and thus positively charged) peptide or protein may be used as neutralizing agent. Exemplarily, the neutralizing agent may be polyethylenimine (PEI) (of arbitrary chain length), poly-l-lysine, protamine, chitosan, a histone sub-unit or an adenoviral core protein. In case of positively charged molecules, as e.g. streptavidin, in particular a polyanionic molecule, for example hyaluronic acid, dextran or long-chain fatty acids may be used as neutralizing agent.

The mixing ratio of charged, in particular negatively charged, molecule and neutralizing agent can be 1:0.05-1.2 (g/g), in particular 1:0.8-1 (g/g). This leads to an advantageous neutralization of the surface charge.

Step d) may comprise an incubating of the charged molecule with the neutralizing agent. The incubating may e.g. occur during a period of 30 seconds to 120 minutes, in particular during a period of 1 minute to 60 minutes.

Regarding the above described methods, before step e), an addition of cations may occur in order to stabilize the complex. Such a stabilizing of complex A increases the transfer efficiency. The addition of cations may occur during and/or after implementation of step d).

The cations may be monovalent, divalent or trivalent cations. Examples for possible cations are Na⁺, K⁺, Ca²⁺, Mg²⁺, and Fe³⁺.

The cations may in particular be added with a concentration of 0 to 1 mM.

Regarding the above described methods, step d) may comprise the addition of albumin.

Surprisingly, it had been determined that the addition of albumin may clearly increase the transfer characteristic of the charged molecules. This is in particular astonishing, as regarding already known systems with fusogenic liposomes, the presence of proteins, e.g. albumin, has a clearly negative influence to the fusogeneity and, thus, the transfer efficiency. The albumin may be added in a concentration of 10 μM to 5 mM, in particular of 10 to 500 μM.

Regarding the above described methods, before, during and/or after step e), a lipid membrane destabilizing agent may be added. The addition may occur after step d). Thus, it may e.g. be added to the fusogenic liposome before incubating with complex A. The addition may also occur during the incubating process or thereafter. Thus, the afterwards occurring fusion is facilitated.

The lipid membrane destabilizing agent may be a detergent. It may in particular have a head-to-chain aspect ratio of more than 1:1, in particular of at least 2:1. With such head-to-chain aspect ratios the destabilizing effect of the detergent increases advantageously.

The lipid membrane destabilizing agent may be neutrally charged. Examples of a lipid membrane destabilizing agent are triton X-100 (C₁₄H₂₂O(C₂H₄O)ₙ), Tween 20 (polysorbate 20) and octyl glucopyramoside.

The concentration of the destabilizing agent may preferably be below its critical micelle concentration (CMC). This reduces the risk that the fusogenic liposomes are dissolved in their structure.

Regarding the above described methods, step b) and/or step e) may comprise an implementation of an ultrasonic treatment and/or an implementation of a high-pressure homogenization. In step b), the ultrasonic treatment or the high-pressure homogenization support the generation of a homogeneous suspension. In step e), the embedding of complex A into the fusogenic liposome is significantly improved by the ultrasonic treatment or the high-pressure homogenization.

The ultrasonic treatment may be implemented at a frequency of 20 to 70 kHz, in particular of 25 to 50 kHz. It may occur during a period of 1 to 60 minutes, in particular 5 to 30 minutes. The implementation may be 50 to 1200 W, in particular 50 to 1000 W.

Regarding the described methods, after step e), a dilution with a buffer with an osmolarity of 200 mOsm or higher may occur. Basically, the buffer may be a watery buffer. The buffer may be a cell culture medium (with or without additions, e.g. serum, antibiotic).

The buffer may have a pH-value of 5 to 10, in particular of 7 to 9. This leads to improved fusogeneities and transfer efficiencies. Exemplary buffers are PBS (phosphate buffered saline), TBS (Tris buffered saline), MOPS (3-(N-morpholino)propane sulphonic acid buffer, carbonate buffer or HBSS (Hank's balanced salt solution) with an osmolarity of 300 mOsm+/−10%.

The dilution may occur in a range from 1:10 to 1:250 (complex A liposomes:buffer), in particular 1:10 to 1:200.

During the dilution and/or after the dilution, an ultrasonic treatment and/or a pressurization may be implemented. It has emerged that such an ultrasonic treatment and/or pressurization avoids an aggregation of the system of the fusogenic complex A liposomes and increases the number of active liposomes. Thereby, the efficiency and homogeneity of the transfer may be improved. For the ultrasonic treatment, the method parameter of the ultrasonic treatment mentioned already above may be used individually or in combination.

Regarding the above described methods, molecule A and/or molecule C may be a lipid or a lipid analogon. A lipid analogon is a lipid, which may not only be formed from nature; it is artificially produced. Lipids and lipid analoga turned out to be explicitly advantageous for the implementation of fusion.

Molecule A may have a C₁₀-C₃₀-proportion in it hydrophobic range. This may have double bonds. Such double bonds may increase the elasticity of the membrane of the arising liposome that leads to a facilitating of the fusion of the liposome with the cell membrane. Suitable examples for molecule A are
A1: 1.2-dioleoyl-3-trimethylammonium-propan chlorides (DOTAP)
A2: N-(2.3-Dioleyloxypropyl)-N, N, N-trimetyl ammonium chlorides (DOTMA)
A3 dimetyl-dioctadecyl ammonium bromides (DDAB)
A4: (1-[2-(oleoyloxy)ethyl]-2-oleyl-3-(2-Hydroxyethyl)imidazolinium chlorides (DOTIM).
A5: β-[N—(N′,N′-dimethylaminoethane)-carbamoyl]cholesterol hydrochlorides
(DC-cholesterol)
A6: 1,2-dilauroyl-sn-glycero-3-ethylphosphocholines (chloride salt) (EPC)
A7: N1-[2-((1S)-1-[(3-aminopropyl)amino]-4-[di(3-amino-propyl)amino]butylcarboxamido)ethyl]-3,4-di[oleyloxy]-benzamides (MVL5).

The proportion of molecule A in the initial mixture may be up to 95 wt/wt %. Particularly preferable are molecules with the above mentioned characteristics and a critical package parameter (CPP, see below) below 1.5.

The molecule C may have a C₁₀-C₃₀-proportion in its hydrophobic range. This may have double bonds. Preferably, the hydrophilic range as well as the hydrophobic range have a neutral character. This leads to a preferable neutralization of the charge density and the repelling forces between positively charged molecules of molecule type A. This way, a stabilizing of the system is achieved. The proportion of molecule C in the initial mixture may be 60 wt/wt&. Suitable examples for molecule C are e.g.:
C1: phosphatidylethanolamines (e.g. 1.2-dioleoyl-sn-glycero-3-phosphoethanolamines, 1.2-dipalmitoyl-sn-glycero-3-phosphoethanolamines, 1.2-dimiristoyl-sn-glycero-3-phosphoethanolamines, 1.2-dielaidoylsn-glycero-3-phosphoethanolamines, 1.2-diphytanolsn-glycero-3-phosphoethanolamines, 1.2-dilinoleoylsn-glycero-3-phosphoethanolamines),
C2: ceramides (e.g. C18 ceramides (d18:1/18:0)N-stearoyl-D-erythro-sphingosines),
C3: cholesterol.

Preferable for the use as molecule type C are phospholipids, in particular phoethanolamines, glycolipids, in particular ceramides as well as sterols and cholesterol.

Molecule A and/or molecule C may generate a planar or nearly planar bilayer when contacting water. Thereby, the generating of liposomes is allowed in a preferable manner.

A planar bilayer is generated if the critical packaging parameter CPP is 1. A nearly planar bilayer is present, if the critical package parameter is between 0.5 and 1 or between 1 and 1.5. In particular, the critical packaging manner may be between 1 and 1.5.

The critical packaging parameter is defined as CPP=v/al, whereby v is the volume of the hydrophobic/lipophilic chain range, a is the cross-sectional surface of the hydrophilic head range and l is the length of lipophilic chain (see M. Salim et al., Amphiphilic designer nano-carriers for controlled release: from drug delivery to diagnostics, Med. Chem. Commun., 2014, 5, 1602, section 4a).

The aromatic molecule B may itself constitute an aromatic compound or at least contain an aromatic group. Molecule B therefore has a cyclic structure motive (aromatic ring) from conjugated double bonds and/or free electron pairs or unoccupied p-orbitals, which fulfil the Huckel's rule. The aromatic ring may have an electron negativity difference Δχ between covalently bound neighbour atoms of at least 0.4, preferably of 0.5 to 2. This increases the polarizability and leads to a further improved fusion. The proportion of molecule B in the initial mixture may have up to 40 wt/wt %.

Suitable examples for molecule B are

fluorescent dyes or dye-labelled lipids, e.g.:
B1: DiOC₁₈(3)3.3′-dioctadecyloxacarbocyanine perchlorates (DiO),
B2: 1.1′-dioctadecyl-3.3.3′.3′-tetramethylindotricarbocyanine iodides (DiR),
B3: N-(4.4-difluoro-5,7-dimethyl-4-bora-3a.4a-diaza-s-indacene-3-propionyl)-1.2-dihexadecanoyl-sn-glycero-3-phosphoethanolamines, triethylammonium salt (BODIPY FL DHPE),
B4: 2-(4.4-difluoro-5-methyl-4-bora-3a.4a-diaza-s-indacene-3-dodecanoyl)-1-hexadecanoyl-sn-glycero-3-phosphocholines (P-BODIPY 500/510 C₁₂—HPC),
B5: lissamine rhodamine B 1.2-diolenoyl-sn-glycero-3-phosphoethanolamines, triethylammonium salt (LR-DOPE),
B6: lissamine rhodamine B 1.2-Dihexadecanoyl-sn-glycero-3-phosphoethanolamines, triethylammonium salt, (LR-DHPE),
B7: texas red 1.2-dihexadecanoyl-sn-glycero-3-phosphoethanolamines, triethylammonium salt (Texas Red DHPE),
B8: N-(7-nitrobenz-2-oxa-1.3-diazol-4-yl)-1.2-diolenoyl-sn-glycero-3-phosphoethanolamines, triethylammonium salt (NBD-DOPE),
B9: 1.2-dioleoyl-sn-glycero-3-phosphoethanolamine-N-(Carboxyfluorescein), ammonium salt, (fluorescein-DOPE),
B10: 1.2-dioleoyl-sn-glycero-3-phosphoethanolamine-N-(1-pyrenesulfonyl), ammonium salt, (pyrene-DOPE),
B11: 1-hexadecanoyl-2-(1-pyrenedecanoyl)-sn-glycero-3-phosphocholines (3-pyrene C₁₀—HPC),
B12: polyphenols (e.g. resveratrol, curcumin, 5-hydroxyflavon),
B13: vitamins (e.g. vitamin E, vitamin A or vitamin K),
B14: cytostatic drugs (e.g. doxorubicine, paclitaxel).

Other pharmacologically effective aromatic substances are also possible.

By choosing a fluorescent molecule B for the production of fusogenic liposomes, this can easily be verified after fusion in the target membrane. The verification is possible to occur via classic systems as e.g. fluorescent microscopy or flow cytometry. Through this, without any time loss, the fusion necessary for the efficient transfer of charged molecules may be directly verified.

All molecules fulfilling the criteria for the molecule type A may be combined in an arbitrary manner with all molecules fulfilling the criteria for the molecule type B, and with all molecules fulfilling the criteria for the molecule type C, as far as the mixture ratio according to the invention is available.

In particular all combinations of molecules A/B/C listed in the following table are possible as initial mixture, wherein the above abbreviations A1, . . . , A7, B1, . . . B14, C1, C2, C3 are used for the different exemplarily listed molecules:

| | | | | | | |
|---|---|---|---|---|---|---|
| A1/B1/C1 | A2/B1/C1 | A3/B1/C1 | A4/B1/C1 | A5/B1/C1 | A6/B1/C1 | A7/B1/C1 |
| A1/B1/C2 | A2/B1/C2 | A3/B1/C2 | A4/B1/C2 | A5/B1/C2 | A6/B1/C2 | A7/B1/C2 |
| A1/B1/C3 | A2/B1/C3 | A3/B1/C3 | A4/B1/C3 | A5/B1/C3 | A6/B1/C3 | A7/B1/C3 |
| A1/B2/C1 | A2/B2/C1 | A3/B2/C1 | A4/B2/C1 | A5/B2/C1 | A6/B2/C1 | A7/B2/C1 |
| A1/B2/C2 | A2/B2/C2 | A3/B2/C2 | A4/B2/C2 | A5/B2/C2 | A6/B2/C2 | A7/B2/C2 |
| A1/B2/C3 | A2/B2/C3 | A3/B2/C3 | A4/B2/C3 | A5/B2/C3 | A6/B2/C3 | A7/B2/C3 |
| A1/B3/C1 | A2/B3/C1 | A3/B3/C1 | A4/B3/C1 | A5/B3/C1 | A6/B3/C1 | A7/B3/C1 |
| A1/B3/C2 | A2/B3/C2 | A3/B3/C2 | A4/B3/C2 | A5/B3/C2 | A6/B3/C2 | A7/B3/C2 |
| A1/B3/C3 | A2/B3/C3 | A3/B3/C3 | A4/B3/C3 | A5/B3/C3 | A6/B3/C3 | A7/B3/C3 |
| A1/B4/C1 | A2/B4/C1 | A3/B4/C1 | A4/B4/C1 | A5/B4/C1 | A6/B4/C1 | A7/B4/C1 |
| A1/B4/C2 | A2/B4/C2 | A3/B4/C2 | A4/B4/C2 | A5/B4/C2 | A6/B4/C2 | A7/B4/C2 |
| A1/B4/C3 | A2/B4/C3 | A3/B4/C3 | A4/B4/C3 | A5/B4/C3 | A6/B4/C3 | A7/B4/C3 |
| A1/B5/C1 | A2/B5/C1 | A3/B5/C1 | A4/B5/C1 | A5/B5/C1 | A6/B5/C1 | A7/B5/C1 |
| A1/B5/C2 | A2/B5/C2 | A3/B5/C2 | A4/B5/C2 | A5/B5/C2 | A6/B5/C2 | A7/B5/C2 |
| A1/B5/C3 | A2/B5/C3 | A3/B5/C3 | A4/B5/C3 | A5/B5/C3 | A6/B5/C3 | A7/B5/C3 |
| A1/B6/C1 | A2/B6/C1 | A3/B6/C1 | A4/B6/C1 | A5/B6/C1 | A6/B6/C1 | A7/B6/C1 |
| A1/B6/C2 | A2/B6/C2 | A3/B6/C2 | A4/B6/C2 | A5/B6/C2 | A6/B6/C2 | A7/B6/C2 |
| A1/B6/C3 | A2/B6/C3 | A3/B6/C3 | A4/B6/C3 | A5/B6/C3 | A6/B6/C3 | A7/B6/C3 |
| A1/B7/C1 | A2/B7/C1 | A3/B7/C1 | A4/B7/C1 | A5/B7/C1 | A6/B7/C1 | A7/B7/C1 |
| A1/B7/C2 | A2/B7/C2 | A3/B7/C2 | A4/B7/C2 | A5/B7/C2 | A6/B7/C2 | A7/B7/C2 |
| A1/B7/C3 | A2/B7/C3 | A3/B7/C3 | A4/B7/C3 | A5/B7/C3 | A6/B7/C3 | A7/B7/C3 |
| A1/B8/C1 | A2/B8/C1 | A3/B8/C1 | A4/B8/C1 | A5/B8/C1 | A6/B8/C1 | A7/B8/C1 |
| A1/B8/C2 | A2/B8/C2 | A3/B8/C2 | A4/B8/C2 | A5/B8/C2 | A6/B8/C2 | A7/B8/C2 |
| A1/B8/C3 | A2/B8/C3 | A3/B8/C3 | A4/B8/C3 | A5/B8/C3 | A6/B8/C3 | A7/B8/C3 |
| A1/B9/C1 | A2/B9/C1 | A3/B9/C1 | A4/B9/C1 | A5/B9/C1 | A6/B9/C1 | A7/B9/C1 |
| A1/B9/C2 | A2/B9/C2 | A3/B9/C2 | A4/B9/C2 | A5/B9/C2 | A6/B9/C2 | A7/B9/C2 |
| A1/B9/C3 | A2/B9/C3 | A3/B9/C3 | A4/B9/C3 | A5/B9/C3 | A6/B9/C3 | A7/B9/C3 |
| A1/B10/C1 | A2/B10/C1 | A3/B10/C1 | A4/B10/C1 | A5/B10/C1 | A6/B10/C1 | A7/B10/C1 |
| A1/B10/C2 | A2/B10/C2 | A3/B10/C2 | A4/B10/C2 | A5/B10/C2 | A6/B10/C2 | A7/B10/C2 |
| A1/B10/C3 | A2/B10/C3 | A3/B10/C3 | A4/B10/C3 | A5/B10/C3 | A6/B10/C3 | A7/B10/C3 |
| A1/B11/C1 | A2/B11/C1 | A3/B11/C1 | A4/B11/C1 | A5/B11/C1 | A6/B11/C1 | A7/B11/C1 |
| A1/B11/C2 | A2/B11/C2 | A3/B11/C2 | A4/B11/C2 | A5/B11/C2 | A6/B11/C2 | A7/B11/C2 |
| A1/B11/C3 | A2/B11/C3 | A3/B11/C3 | A4/B11/C3 | A5/B11/C3 | A6/B11/C3 | A7/B11/C3 |
| A1/B12/C1 | A2/B12/C1 | A3/B12/C1 | A4/B12/C1 | A5/B12/C1 | A6/B12/C1 | A7/B12/C1 |
| A1/B12/C2 | A2/B12/C2 | A3/B12/C2 | A4/B12/C2 | A5/B12/C2 | A6/B12/C2 | A7/B12/C2 |
| A1/B12/C3 | A2/B12/C3 | A3/B12/C3 | A4/B12/C3 | A5/B12/C3 | A6/B12/C3 | A7/B12/C3 |
| A1/B13/C1 | A2/B13/C1 | A3/B13/C1 | A4/B13/C1 | A5/B13/C1 | A6/B13/C1 | A7/B13/C1 |
| A1/B13/C2 | A2/B13/C2 | A3/B13/C2 | A4/B13/C2 | A5/B13/C2 | A6/B13/C2 | A7/B13/C2 |
| A1/B13/C3 | A2/B13/C3 | A3/B13/C3 | A4/B13/C3 | A5/B13/C3 | A6/B13/C3 | A7/B13/C3 |
| A1/B14/C1 | A2/B14/C1 | A3/B14/C1 | A4/B14/C1 | A5/B14/C1 | A6/B14/C1 | A7/B14/C1 |
| A1/B14/C2 | A2/B14/C2 | A3/B14/C2 | A4/B14/C2 | A5/B14/C2 | A6/B14/C2 | A7/B14/C2 |
| A1/B14/C3 | A2/B14/C3 | A3/B14/C3 | A4/B14/C3 | A5/B14/C3 | A6/B14/C3 | A7/B14/C3 |

Thereby, “A1/B1/C1” represents for example the mixture DOTAP/DiO/phosphatidylethanolamine or “A3/B4/C2” for the mixture DDAB/P-BODIPY 500/510 C₁₂—HPC/C18-ceramide.

The invention furthermore provides a fusion mixture available according to one of the above described methods.

Moreover, the invention provides the use of such a fusion mixture for fusion with a lipid membrane. The lipid membrane may be a biological membrane (or a compound thereof) or an artificially produced lipid membrane. Thereby, the lipid membrane may in particular be a cell membrane, including plasm or cell organelle membranes, a compound of a cell membrane, a purified biological membrane or also an artificial membrane system.

The invention also provides a method for transfer of a charged molecule and/or through a lipid membrane by means of fusion with the steps:
implementation of one of the above described methods for producing a fusion mixture,
bringing into contact the fusion mixture with a lipid membrane so that the fusion mixture fuses with the lipid membrane.

Surprisingly it turned out that the complex A liposomes generated with the above described method directly after contact fuse with the target membrane and emit the complex A into the lumen covered by the target membrane.

During the method, the lipid membrane may be a biological membrane (or a compound thereof) or an artificially produced lipid membrane. The lipid membrane may be a cell membrane including plasm- or cell organelle membranes, a compound of a cell membrane, a purified biological membrane or also an artificial membrane system.

The step of brining into contact may be implemented in suspension and/or with a lipid membrane being adhered to a substrate surface. The step of bringing into contact may occur for a period of 1 minute to 60 minutes, in particular 5 to 30 minutes. In this step, the temperature may be in a range of 20 to 45° C. The pH-value may be in the range of 6 to 9. The osmolarity may have values of at least 200 mOsm.

### BRIEF DESCRIPTION OF THE DRAWINGS

In the following, the invention is illustrated in detail by means of the embodiments and the enclosed Figures. Thereby illustrates and/or shows:

FIG. 1A: the fusogeneity of classic fusion systems with increasing concentration of charged molecules (DNA);

FIG. 1B: the fusogeneity of classic fusion systems with increasing concentration of charged particles;

FIG. 2A: the influence of the neutralization of charged molecules (DNA) by means of peptide to the fusogeneity of fusogenic liposomes as well as the transfer efficiency;

FIG. 2B: the influence of the neutralization of charged molecules (RNA) by means of peptide to the fusogeneity of fusogenic liposomes as well as the transfer efficiency;

FIG. 3: the influence of the neutralization of charged molecules (RNA) by means of polyprotonated polymers to the fusogeneity and transfer efficiency;

FIG. 4: the zeta potential switching subject to the complex composition;

FIG. 5: the influence of the membrane destabilization to the fusogeneity and transfer of charged molecules;

FIG. 6: the influence of additional cations to the fusogeneity and transfer of charged molecules;

FIG. 7: the influence of the pH-value of the liposome buffer to the fusogeneity and transfer of charged molecules;

FIGS. 8A and 8B: the influence of the pH-value of the dilution buffer (PBS) to the fusogeneity and transfer of charged molecules;

FIG. 9: the dependence of the fusogeneity and transferability of charged molecules of the composition of fusogenic liposomes;

FIGS. 10A and 10B: the universal transferability of charged molecules into different cell lines and primary cells by means of adjusted fusion;

FIG. 11: the influence of additional ultrasonic treatment to the fusogeneity and transfer of charged molecules;

FIG. 12: a comparison of conventional fusogenic liposomes and a fusion system according to the invention;

FIG. 13: different lipid compositions (components A, B, and C) of functional fusogenic liposomes;

FIG. 14A: the influence of albumins to the transfer of charged molecules through the example of DNA;

FIG. 14B: the influence of albumins to the transfer of charged molecules through the example of RNA.

### DETAILED DESCRIPTION

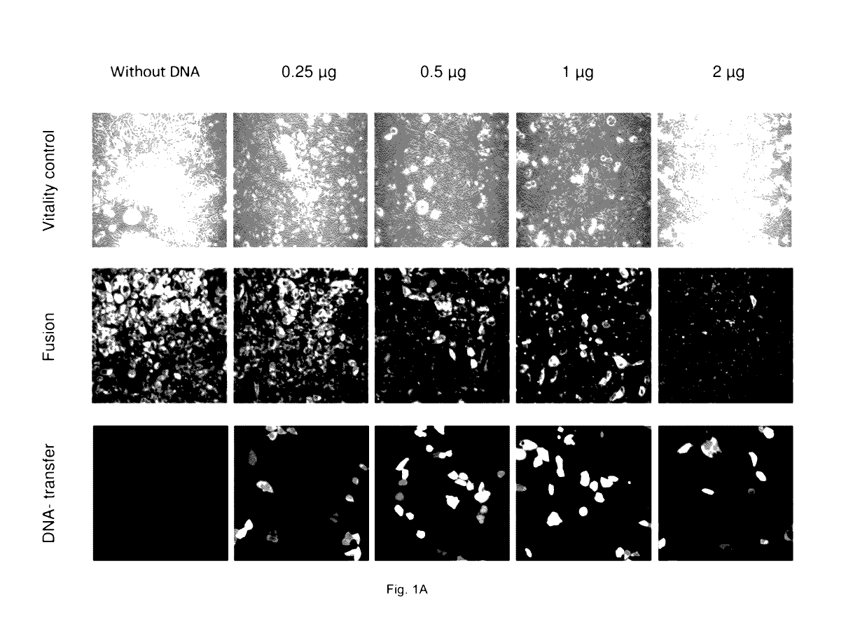

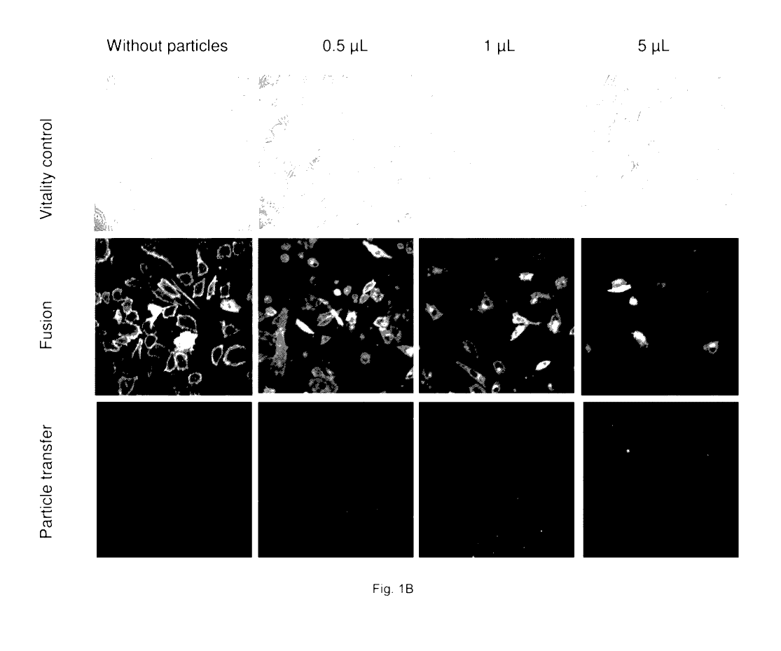

FIG. 1A illustrates the strongly reduced fusogeneity of classic fusion systems (no neutralization of charged molecules, no puffer adjustments, no additional ions, no pH-value adjustments, no additional ultrasonic treatment) at increasing concentration of charged molecules (DNA). As DNA exemplarily also in the following examples, a construction had been used, which after functional insertion of the cells is translated into a green fluorescent protein (GFP) and, thus, may easily be detected by means of microscopy. For the verification of the transfer efficiency of charged molecules through classic, fusogenic liposomes of the composition: positively charged lipid (DOTAP), fusogenic molecule (DiR) and neutral lipid (DOPE) in the weight ratio 1:0.1:1, 10 μl of a 3 mM solution had been used and incubated with increasing concentration at the DNA. The incubation occurred in 20 mM HEPES (2-(4-(2-hydroxyethyl)-1-piperazinyl)-ethansulphonic acid) pH 7.4 at room temperature (RT).

Subsequently, the fusogenic lipidic DNA liposomes were treated in the ultrasonic bath at 36 kHz and 200 W for 20 min at RT in order to obtain fusogenic liposomes of middle size of approximately 340 nm. 10 μl of the arising fusogenic lipid-DNA-liposomes were again diluted with PBS (phosphate buffered salt solution) in the ratio of 1:50 and again incubated in the ultrasonic bath as before under same conditions, in order to obtain a possibly large number of fusogenic liposomes. The diluted liposomes were added to adhered CHO (Chinese hamster ovary) cells instead of the cell culture medium, which beforehand had been seeded in a density of 15,000 cells per cm² and incubated for 20 min at 37° C.

Fusion was stopped by exchanging the fusion solution against cell culture medium. 24 hours after termination of the fusion reaction, the fusion efficiency (middle) was verified by means of fluorescence microscopy as well as the functional transfer of the plasmid by means of protein expression analysis (below). The strong reduction of the fusogeneity of the liposomes is recognizable with increasing concentration of charged molecule. Simultaneously, for none concentration a functional transfer of the DNA occurs (i.e. expression of coding sequence and, thus, generation of the green fluorescent protein (light signal) with an efficiency of more than 10%.

FIG. 1B illustrates the strongly reduced fusogeneity of classic fusion systems with increasing concentration of charged particles (positively charged nanoballs). For verifying the transfer efficiency of charged particles by classic, fusogenic liposomes of the composition: positively charged lipid (DOTAP), fusogenic molecule (DiR) and neutral lipid (DOPE) in the weight ratio 1:0.1:1, 10 μl of a 3 mM solution were used and incubated with increasing concentrations of positively charged nanoballs. The incubation occurred in 20 mM HEPES pH 7.4 for 10 min at RT. Unless indicated otherwise, the basic composition of the fusogenic liposomes (DOTAP/DiR/DOPE) was maintained also for the following embodiments and Figures.

Subsequently, the fusogenic lipid-ball-liposomes were treated in the ultrasonic bath at 36 kHz and 200 W for 20 min at RT in order to obtain fusogenic liposomes of middle size of approximately 340 nm. 10 μl of the arising fusogenic lipid-ball-liposomes were diluted with PBS in a ratio of 1:50 and again incubated for 20 min under same conditions as beforehand in the ultrasonic bath in order to obtain a possible large number of fusogenic liposomes. The diluted liposomes were given instead of the cell culture medium to the adherent CHO (Chinese hamster ovary) cells, which had been seeded one day before in a density of 15,000 cells per cm² and incubated for 20 min at 37° C.

Fusion was stopped through exchange of fusion solution against cell culture medium. Directly after termination of the fusion reaction, the fusion efficiency (middle) was verified by means of fluorescence microscopy as well as the functional transfer of the plasmid by means of protein expression analysis (below). The strong reduction of the fusogeneity of the liposomes is to be noted with increasing concentration of charged particles. Simultaneously, nearly no transfer of charged particles takes place.

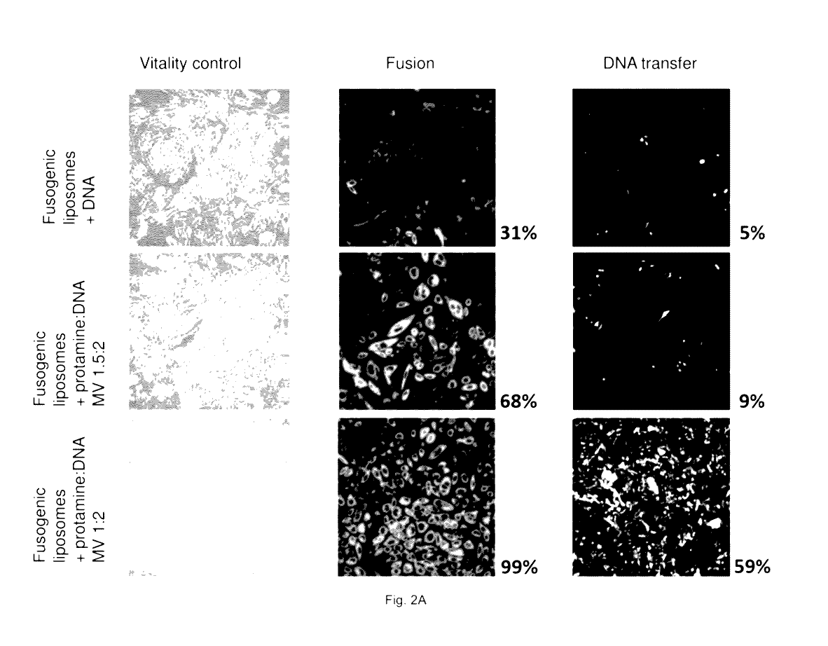

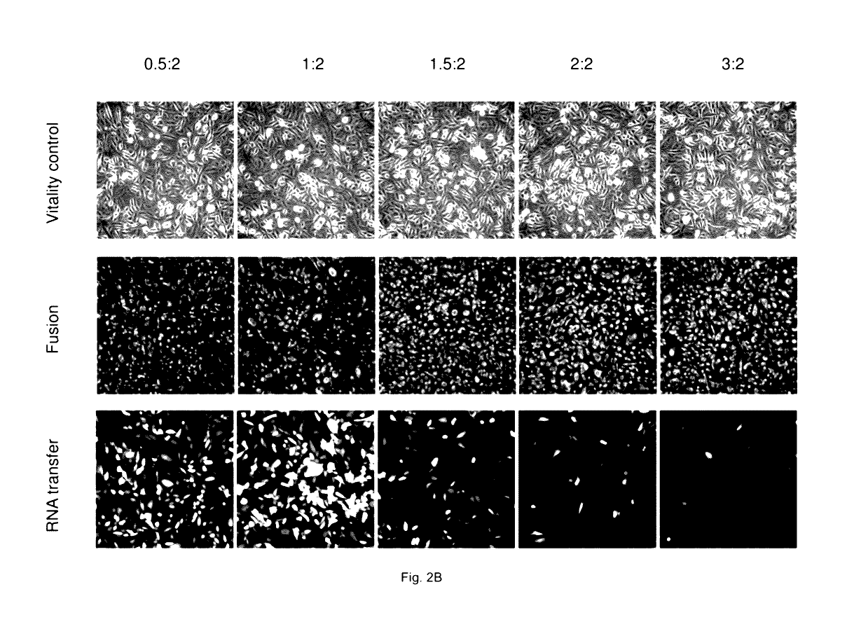

FIG. 2A illustrates the influence of the neutralization of charged molecules (DNA) by means of peptide to the fusogeneity of fusogenic liposomes as well as the transfer efficiency. For producing the complex from DNA and protamine as complex creator (complex A) per preparation of 2 μg cDNA either without (“fusogenic liposomes+DNA”, top) or with defined concentrations of polycationic substance (protamine) as neutralizing agent in the weight ratio (protamine:DNA) 1.5:2 (middle) and 1:2 (below) was implemented in the fusion reaction. As solution buffer 5 μl Tris-buffer (Tris(hydroxymethyl)-aminomethane) was used (10 mM Tris, pH 7.5). The incubation in solution buffers occurred during a 20 minutes incubation at RT. During this time, 10 μl of a 3 mM fusogenic lipidic mixture was suspended in the ultrasonic bath at 45 kHz and 70 W for 10 min at RT in order to obtain fusogenic liposomes of middle size of approximately 340 nm.

The lipid composition of the fusogenic liposomes consisted of positively charged lipid, fusogenic molecule, and neutral lipid in a weight ratio 1:0.1:1 in 20 mM HEPES, pH 7.4. After termination of the incubation times, 1 μl triton buffers (10 μM triton X-100, 10 μM NaCl, 2 μM TrisHCl pH 7.6) were added to the fusogenic liposomes and subsequently merged with the finished complex A and treated for 20 min at 45 kHz and RT in the ultrasonic bath. 10 μl of the arising fusogenic complex A liposomes were diluted with PBS in the ratio 1:50 and again incubated for 20 min under same conditions as before in the ultrasonic bath in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against the cell culture medium. 24 hours after termination of the fusion reaction, by means of fluorescent microscopy and flow cytometry, the fusion efficiency as well as the functional transfer of the plasmid were verified by means of protein expression analysis. The cells identified as positive in the flow cytometry (red fluorescent due to the red fluorescent DiR contained in the fusion mixture as well as additionally green fluorescent after translation of the introduced DNA in green fluorescent protein (GFP)) are indicated in percent next to the Figures.

FIG. 2B illustrates the influence of the neutralization of the charged molecules (RNA) by means of peptide to the fusogeneity of fusogenic liposomes as well as the transfer efficiency. For producing the complex from RNA and complex creator (complex A), per preparation 2 μg RNA for expression of GFP (green fluorescent protein) with defined concentrations of neutralizing polycationic substance (protamine) in the indicated weight ratios (protamine:RNA) were implemented in the fusion reaction. As solution buffer, 5 μl buffer was used (10 mM Tris, pH 7.5). The incubation in the solution buffer occurred during a 20 minutes incubation at RT. During this time, 10 μl of a 3 mM fusogenic lipidic mixture was suspended in the ultrasonic bath at 36 kHz and 70 W for 10 min at RT in order to obtain fusogenic liposomes of middle size of approximately 340 nm.

The lipid composition of the fusogenic liposomes consisted of positively charged lipid, fusogenic molecule, and neutral lipid in a weight ratio 1:0.1:1 in 20 mM HEPES, pH 7.4. After termination of the incubation times, 100 μM NaCl were added to the fusogenic liposomes and treated with finished complex A for 20 min at 36 kHz and RT in the ultrasonic bath. 10 μl of the arising fusogenic complex A liposomes were diluted with PBS in the ratio 1:50 and again incubated or 20 min under same conditions as before in the ultrasonic bath in order to obtain a possible large number of fusogenic liposomes.

The diluted complex A liposomes were added instead of the cell culture medium to adherent CHO (Chinese hamster ovary) cells, which had been seeded the day before in a density of 15,000 cells per cm2 and incubated for 20 min at 37° C. Fusion was stopped by exchange of the fusion solution against cell culture medium. 3 hours after termination of the fusion reaction, the fusion efficiency was verified by means of fluorescent microscopy as well as the expression of the mRNA by means of protein expression analysis. Clearly visible is in particular the preferable RNA transfer at 0.5:2 as well as 1:2 ratios of RNA:protamine, while the fusion efficiency is hardly influenced by different mixture ratios.

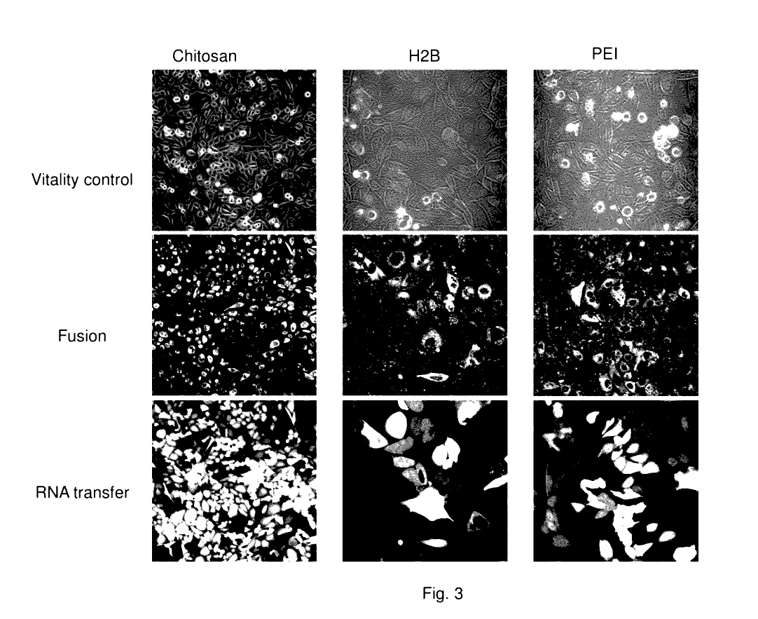

FIG. 3 illustrates the influence of the neutralization of charged molecules (RNA) by means of polyprotonated polymers to fusogeneity and transfer efficiency. For producing the complex of DNA and complex creator (complex A), per preparation 2 μg DNA were incubated for expression of GFP (green fluorescent protein) with 1 μg poly-ethylenimine (PEI), 1 μg H2B histone protein and/or 0.5 μg chitosan as polycationic substance as alternative to protamine. As solution buffer, 5 μl buffer (10 mM Tris, pH 7.5) were used. The incubation in solution buffers occurred during a 20 minutes incubation at RT. During this time, 10 μl of 3 mM fusogenic lipidic mixture were suspended in the ultrasonic bath at 45 kHz and 70 W for 10 min at RT in order to obtain fusogenic liposomes of middle size of approximately 340 nm.

The lipid composition of the fusogenic liposomes consisted of positively charged lipid, fusogenic molecule and neutral lipid in the weight ratio 2:0.2:1 in 20 mM HEPES, pH 7.4. After termination of the incubation times, 50 μM NaCl were added to the fusogenic liposomes and treated with the finished complex A for 20 min at 45 kHz and RT in the ultrasonic bath. 10 μl of the arising fusogenic complex A liposomes were diluted with PBS at a ratio 1:50 and again incubated for 20 min under same conditions as before in the ultrasonic bath in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against cell culture medium. 3 hours after termination of the fusion reaction, the fusion efficiency was verified by means of fluorescent microscopy as well as the functional expression of the mRNA by means of protein expression analysis. The following fusion as well as functional transfer of the charged molecules also regarding PEI and/or H2B complexation is to be noted.

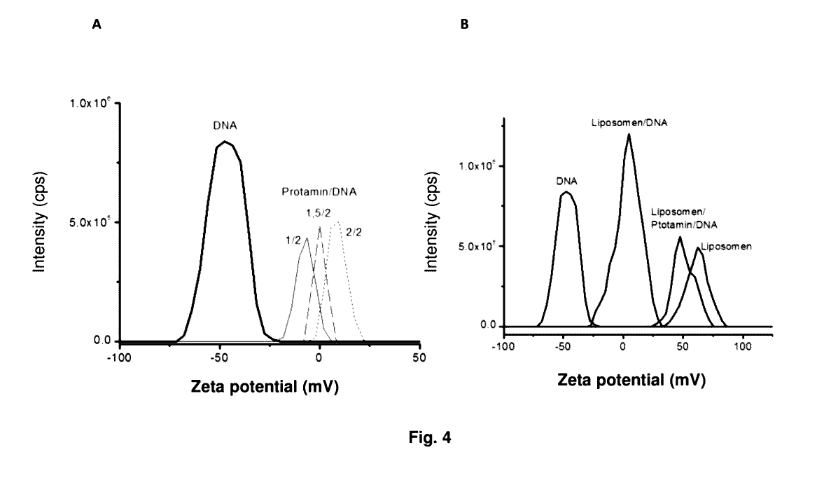

FIG. 4 illustrates the zeta potential switching subject to the complex composition. The zeta potential of DNA for the left diagram was determined by the fact that 2 μg DNA was absorbed in a water/PBS mixture (950 μl distilled water and 50 μl PBS). A zeta potential of about −50 mV is shown.

If the identical quantity of DNA in different ratios was complexed with protamine for 0.5 hours at RT (1; 1.5 and 2 μg protamine corresponds to protamine/DNA ratios of 1/2; 1.5/2 and 2/2), the zeta potential of the complexes is clearly influenced. With increasing protamine concentration, the zeta potential of the complexes is switched to positive ranges. The protamine/DNA ratio is preferably adjusted in a manner that the zeta potentials of the complexes compared to DNA are clearly reduced and nevertheless the complexes still electrostatically interact in an ideal manner with the positively charged liposomes (the zeta potential of which thereby remain in the negative range). In this example, the complex with the 1/2 protamine/DNA ratio corresponds to these criteria.

In order to demonstrate the dependency of transfection efficiency and zeta potential, for the left diagram B, liposomes as above described generated with a concentration of 3 mM in 20 mM HEPES buffer and 1/100 diluted in a water/PBS mixture (940 μl distilled water and 50 μl PBS). 10 μl of these liposomes before the dilution were either only with 2 μg DNA or 2 μg DNA neutralized with 1 μg protamine (1/2) incubated and, like 2 μg DNA, 1/100 diluted. The liposomes show a positive zeta potential (app. 60 mV), while the DNA ranged in the negative range (−50 mV).

With incubation of the liposomes with protamine complexed DNA, their positive zeta potential was clearly less reduced (50 mV) than without protamine (10 mV). A zeta potential in the range of 50 mV, thus, is still adequate for a successful fusion and DNA transfer, while a lower zeta potential of the complexes does not lead to the fusion.

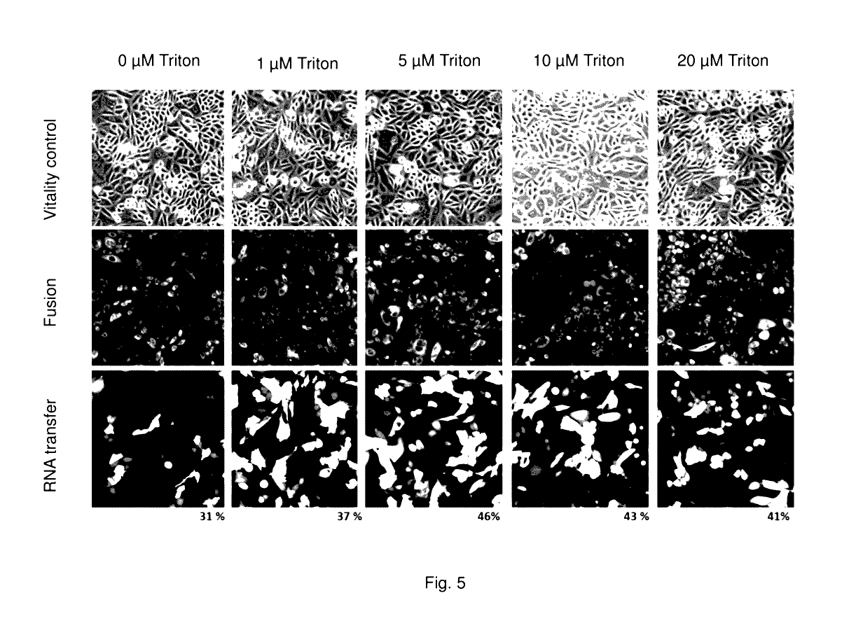

FIG. 5 illustrates the influence of the membrane destabilization to fusogeneity and transfer of charged molecules. For producing the complex of DNA and complex creator (complex A), per preparation 2 μg cDNA for the expression of GFP (green fluorescent protein) with polycationic substance (protamine) at a weight ratio 2:1 were incubated. As solution buffer, 5 μl buffer were used (10 mM Tris, pH 7.5). Before incubation for 20 min at RT, Triton X-100 was added to the preparations in the concentrations 0, 1, 5, 10, and 20 μM. During this time, 10 μl of a 3 mM fusogenic lipidic mixture was suspended in the ultrasonic bath at 45 kHz and 70 W for 10 min at RT in order to obtain fusogenic liposomes of a middle size of approximately 340 nm.

The lipid composition of the fusogenic liposomes consisted of positively charged lipid, fusogenic molecule and neutral lipid at a weight ratio 1:0.1:1 in 20 mM HEPES, pH 7.4. After termination of the incubation times, the fusogenic liposomes were incubated with the finished complex A and treated for 20 min at 45 kHz and RT in the ultrasonic bath. 10 μl of the arising fusogenic complex A liposomes were diluted and again incubated for 20 min under same conditions as before in the ultrasonic bath in order to obtain a possible large number of fusogenic liposomes.

The diluted complex A liposomes were added instead of the cell culture medium to the adherent HeLa cells, which had been seeded the day before in a density of 15,000 cells per cm² and incubated for 20 min at 37° C. HeLa cells were used as these cells have only a lowered fusion efficiency and DNA transfer rate. Fusion was stopped by exchange of the fusion solution against cell culture medium. 24 hours after termination of the fusion reaction the fusion efficiency was verified by means of fluorescent microscopy and flow cytometry as well as the functional transfer of the plasmid by means of protein expression analysis. The cells in the flow cytometry positively identified cells are indicated in percent. It is to be noted that cells with naturally lowered fusion efficiency in the presence of low concentration of membrane destabilizing molecules may be charged as fused as well as with charged molecules.

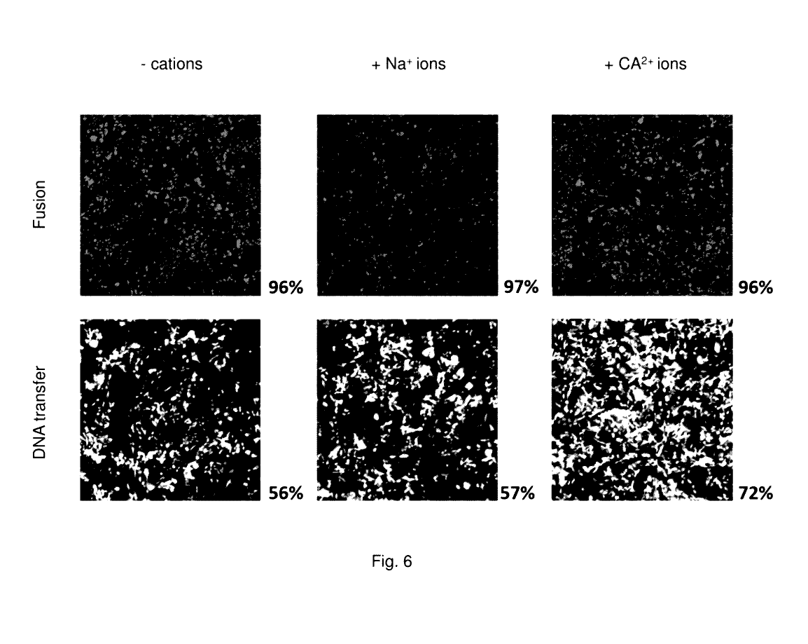

FIG. 6 illustrates the influence of additional cations to the fusogeneity and transfer of charged molecules. For producing the complex of DNA and complex creator (complex A) per preparation, 2 μg cDNA for expression of GFP (green fluorescent protein) with polycationic substance (protamine) at a weight ratio 2:1 were incubated. As solution buffer, 5 μl buffer were used (10 mM Tris, pH 7.5). Additionally, in a preparation Na⁺-ions were added (100 mM NaCl). The incubation in the solution buffer occurred during a 20 minutes incubation at RT. During this time, 10 μl of 3 mM fusogenic lipid mixture were suspended in the ultrasonic bath at 36 kHz and 70 W for 10 min at RT in order to obtain fusogenic liposomes of middle size of approximately 340 nm.

The lipid composition of the fusogenic liposomes consisted of positively charged lipid, fusogenic molecule and neutral lipid at a weight ratio 1:0.1:1 in 20 mM HEPES, pH 7.4. After termination of the incubation times, fusogenic liposomes were incubated with the finished complex A and treated for 20 min at 45 kHz and RT in the ultrasonic bath. 10 μp of the arising fusogenic complex A liposomes were diluted with PBS at a ratio 1:50. In a preparation, which beforehand had been incubated only in 20 mM HEPES as solution buffer, at this point in time, Ca²⁺-ions were added (20 μM CaCl₂). Subsequently, all preparations were again incubated for 20 min under the same conditions as before in the ultrasonic bath in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against cell culture medium. 24 hours after termination of the fusion reaction, the fusion efficiency was verified by means of fluorescent microscopy and flow cytometry as well as the functional transfer of the plasmid by means of protein expression analysis. The cells in the flow cytometry positively identified cells are indicated in percent. While the fusion itself is not changed due to additional cations, these show a positive effect to the functional transfer of charged molecules.

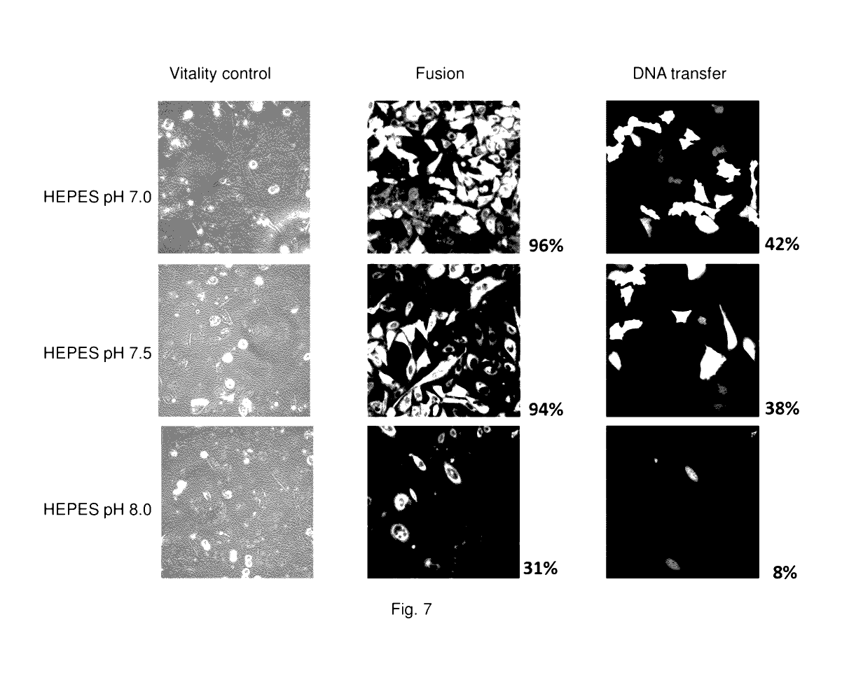

FIG. 7 illustrates the influence of the pH-value of the liposome buffer to the fusogeneity and transmission of charged molecules. For producing the complex of DNA and complex creator (complex A), per preparation 2 μg cDNA for expression of GFP (green fluorescent protein) with polycationic substance (protamine) were incubated at a weight ratio of 2:1. As solution buffer, 5 μl buffer was used (20 mM HEPES, 100 mM NaCl). Per preparation, the pH-value of the buffer was varied in the range of 7.0 via 7.4 to 8.0. The incubation in the solution buffer occurred during a 20 minutes incubation at RT. During this time, 10 μl of 3 mM fusogenic lipid mixture were suspended in the ultrasonic bath at 36 kHz and 70 W for 10 min at RT in order to obtain fusogenic liposomes of middle size of approximately 340 nm.

The lipid composition of the fusogenic liposomes consisted of positively charged lipid, fusogenic molecule and neutral lipid at weight ratios 1:0.1:1 in 20 mM HEPES, pH 7.4. After termination of the incubation times, 1 μl of a 100 mM solution NaCl was added to the fusogenic liposomes and treated with finished complex A for 20 min at 45 kHz and RT in the ultrasonic bath. 10 μl of the arising fusogenic complex A liposomes were diluted with PBS at a ratio 1:50 and again incubated for 20 min under same conditions as before in the ultrasonic bath in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against the cell culture medium. 24 hours after termination of the fusion reaction, the fusion efficiency was verified by means of fluorescent microscopy and flow cytometry as well as the functional transfer of the plasmid by means of protein expression analysis. The cells in the flow cytometry positively identified are indicated in percent. A pH optimum is clearly recognizable in the range of 7 to 7.4. Higher pH-values may hinder the fusion as well as the transfer of charged molecules.

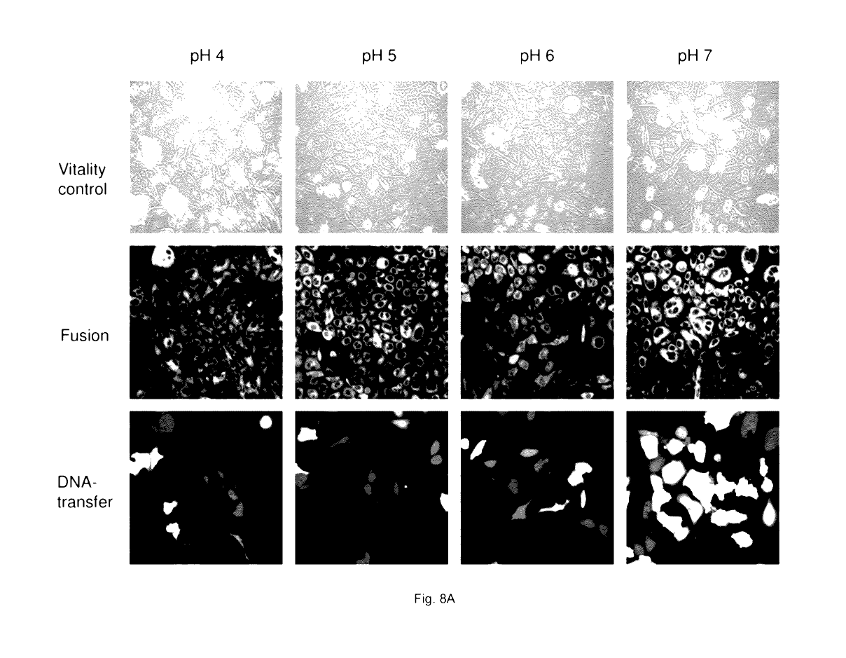

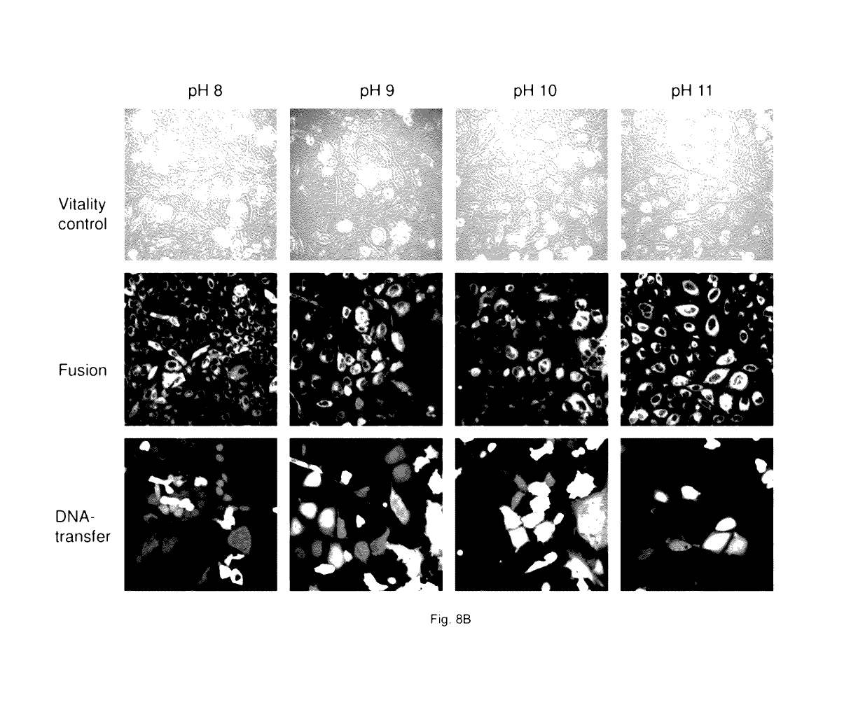

FIGS. 8A and 8B illustrate the influence of the pH-value of the dilution buffer (PBS) after generation of the fusogenic complex A liposome to the fusogeneity and transfer of charged molecules. For producing the complex of DNA and complex creator (complex A), per preparation, 2 μg cDNA for expression of GFP (green fluorescent protein) with polycationic substance (protamine) at a weight ratio 2:1 were incubated. As solution buffer, 5 μl buffer were used (10 mM Tris, pH 7.5). The incubation in the solution buffer occurred for 20 min at RT. During this time, 10 μl of a 3 mM fusogenic lipid mixture were suspended in the ultrasonic bath at 36 kHz and 200 W for 10 min at RT in order to obtain fusogenic liposomes of middle size of approximately 340 nm.

The lipid composition of the fusogenic liposomes consisted of positively charged lipid, fusogenic molecule, and neutral lipid at weight ratios 1:0.1:1 in 20 mM HEPES, pH 7.4. After termination of the incubation times, 1 μl of a 100 mM solution NaCl was added to the fusogenic liposomes with finished complex A and incubated for 20 min at 36 kHz and RT in the ultrasonic bath. 10 μl of the arising fusogenic complex A liposomes were diluted with PBS at a ratio 1:50. The pH-value of the PBS buffer thereby was varied in the range of pH 4 to pH 11. FIG. 8A represents the pH-range of 4 to 7 and FIG. 8B constitutes the pH-range 8 to 11. After dilution, all preparations were again incubated for 20 minutes under same conditions as before in the ultrasonic bath in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against cell culture medium. 24 hours after termination of the fusion reaction, the fusion efficiency was verified by means of fluorescent microscopy as well as the functional transfer of the plasmid by means of protein expression analysis. Particularly preferable is the range around pH 7 to 9 of the functional transfer of charged molecules. Thereby, the transfer is also possible at higher pH-values, however, this comes along with then increasing osmotic stress for the cells. The fusion itself is nearly unchanged in the tested pH range.

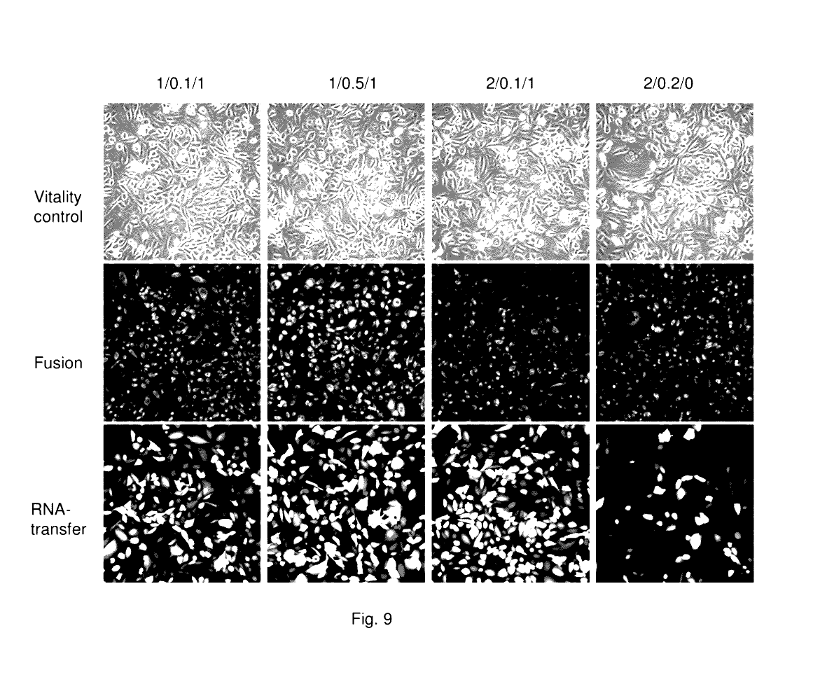

FIG. 9 illustrates the dependency of the fusogeneity and transferability of charged molecules of the composition of the fusogenic liposomes. For producing the complex of mRNA and complex creator (complex A), per preparation 2 μg mRNA for expression of GFP (green fluorescent protein) were incubated with polycationic substance (protamine) at a weight ratio of 2:1. As solution buffer, 5 μl buffer were used (10 mM Tris, pH 7.5). The incubation in the solution buffer occurred during a 20 minutes incubation at RT. During this time, 10 μl of a 3 mM fusogenic lipidic mixture were suspended in the ultrasonic bath at 36 kHz and 200 W for 10 min at RT in order to obtain fusogenic liposomes of middle size of approximately 340 nm.

The lipid composition of the fusogenic liposomes was varied per preparation in a manner that for the compounds positively charged lipid:fusogenic molecule:neutral lipid the weight ratios 1:0.1:1, 1:0.5:1, 2:0.1:1, and 2:0.2:0 in 20 mM HEPES, pH 7.5 as indicated in the Figure were used. After termination of the incubation times, 1 μl of a 100 mM NaCl solution was added to the developed fusogenic liposomes and, respectively, incubated with finished complex A for 20 min at 36 kHz and RT in the ultrasonic bath. 10 μl of the developed fusogenic complex A liposomes were diluted with PBS at a ratio 1:50. After dilution, all preparations were again incubated for 20 min under same conditions as before in order to obtain a possible large number of fusogenic liposomes.

The diluted complex A liposomes were added instead of the cell culture medium to the adherent CHO (Chinese hamster ovary) cells, which had been seeded the day before in a density of 15,000 cells per cm² and incubated for 20 min at 37° C. Fusion was stopped by exchange of the fusion solution against cell culture medium. 3 hours after termination of the fusion reaction, the fusion efficiency was verified by means of fluorescent microscopy as well as the functional expression of the mRNA by means of protein expression analysis. It is clearly recognizable that the procedure according to the invention allows fusion as well as transfer of charged molecules for all tested lipid ratios.

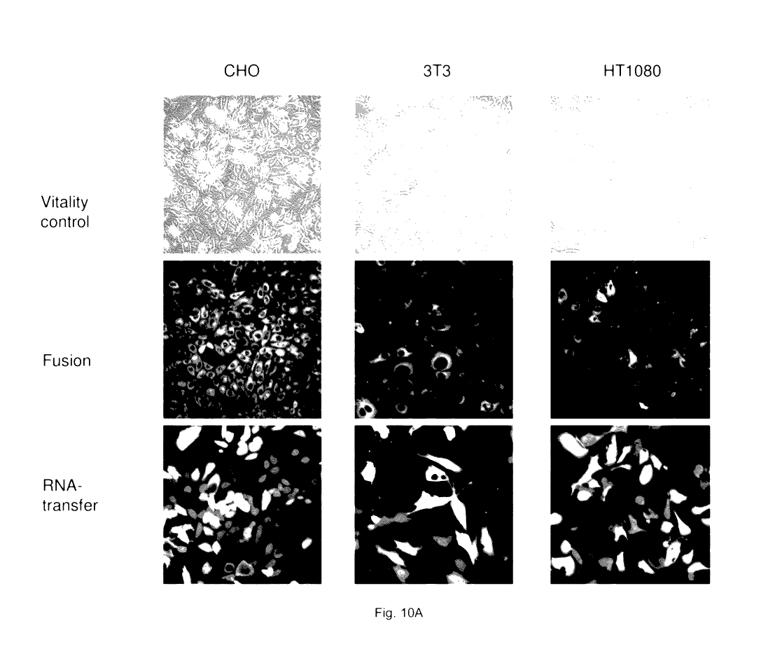

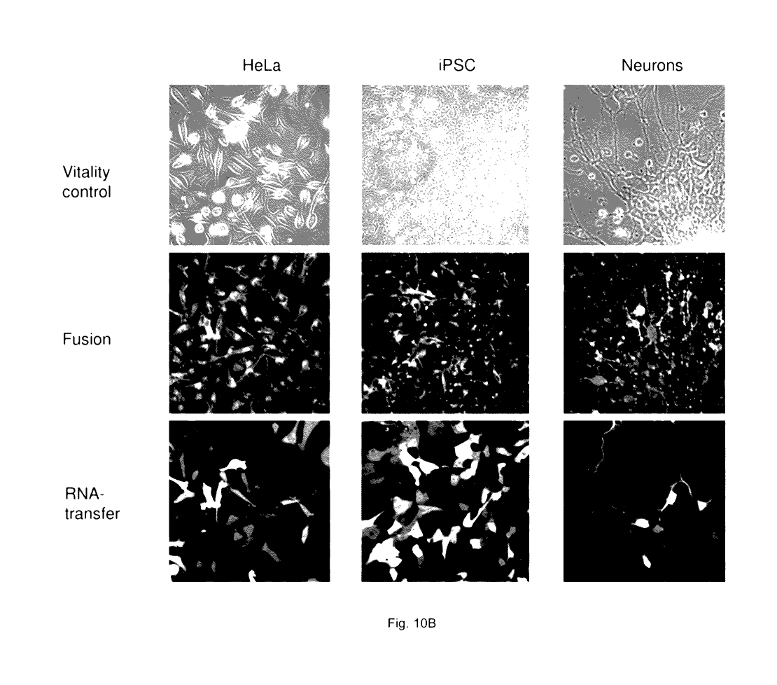

FIGS. 10A and 10B illustrate the universal transferability of charged molecules in different cell lines and primary cells by means of generated fusion mixture according to the invention. For producing the complex from mRNA and complex creator (complex A), per preparation, 2 μg mRNA for expression of GFP (green fluorescent protein) with polycationic substance (protamine) at a weight ratio 2:1 were incubated. As solution buffer, 5 μl buffer were used (10 mM Tris, pH 7.5). The incubation in the solution buffer occurred during a 20 minutes incubation at RT. During this time, 20 μl of a 3 mM fusogenic lipidic mixture was suspended in the ultrasonic bath at 36 kHz and 200 W for 10 min at RT in order to obtain fusogenic liposomes of middle size of approximately 340 nm.

The lipid composition of the fusogenic liposomes consisted of positively charged lipid, fusogenic molecule and neutral lipid at a weight ratio 1:0.1:1 in 20 mM HEPES, pH 7.4. After termination of the incubation times, 1 μl of a 100 mM NaCl solution was added to the developed fusogenic liposomes and incubated with finished complex A for 20 min at 36 kHz and RT in the ultrasonic bath. 10 μl of the developed fusogenic complex A liposomes were diluted with PBS at a ratio 1:50. After dilution, all preparations were again incubated for 20 min under same conditions as before in the ultrasonic bath in order to obtain a possible large number of fusogenic liposomes.

The diluted complex A liposomes were added instead of the cell culture medium to different cell lines and primary cells (CHO (Chinese hamster ovary), 3T3, HT1080, HeLa, iPSC, primary cortical neurons), which had been seeded the day before in a density of 15,000 cells per cm². Dependent on the cell type and, thus, on cell-type specific fusion characteristics, these were incubated for 15 to 30 min at 37° C. with the fusion solution (CHO=15 min, FIG. 10A; 3T3=15 min, FIG. 10A; HT1080=30 min, FIG. 10A; HeLa=30 min, FIG. 10B; iPSC=25 min, FIG. 10B; neurons=20 min, FIG. 10B). Fusion was stopped by exchange of the fusion solution against the cell culture medium. 3 hours after termination of the fusion reaction, the fusion efficiency was verified by means of fluorescent microscopy as well as the functional expression of mRNA by means of protein expression analysis. It is clearly recognizable that in all cases, fusion and DNA transfer has taken place and, thus, the method according to the invention is applicable for producing a fusion mixture universally for animal cell lines and primary cells.

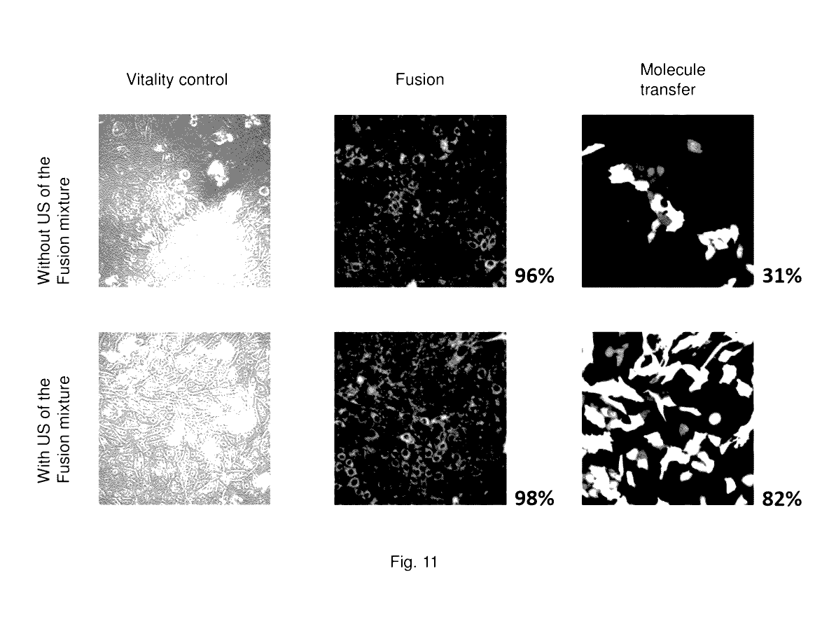

FIG. 11 illustrates the influence of additional ultrasonic treatment to fusogeneity and transfer of charged molecules. For producing the complex of DNA and complex creator (complex A), per preparation, 2 μg cDNA for expression of GFP (green fluorescent protein) with polycationic substance (protamine) at a weight ratio 2:1 were incubated. As solution buffer, 5 μl buffer were used (10 mM Trios, pH 7.5). The incubation in the solution buffer occurred for 10 min at RT. During this time, 10 μl of a 3 mM fusogenic lipid mixture were suspended in the ultrasonic bath at 36 kHz and 200 W for 10 min at RT in order to obtain fusogenic liposomes of middle size of approximately 340 nm.

The lipid composition of the fusogenic liposome consisted of positively charged lipid, fusogenic molecule and neutral lipid at a weight ratio 1:0.1:1 in 20 mM HEPES, pH 7.4 After termination of the incubation times, 1 μl of a 100 mM NaCl and 0.5 μM Triton X-100 solution were added to the fusogenic liposomes and incubated with finished complex A for 20 minutes at 36 kHz and RG in the ultrasonic bath. 10 μl of the arising fusogenic complex A liposomes were diluted with PBS at a ratio of 1:50. During classic fusion liposomes for transfer of uncharged molecules incubate the diluted fusion solution without any further ultrasonic treatment directly with the cells to be fused (without US), the preparation was again incubated after dilution for 20 minutes under same conditions as before in the ultrasonic bath (with US).

The diluted complex A liposomes in the following were added instead of the cell culture medium to the adherent CHO (Chinese hamster ovary) cells, which have been seeded the day before in a density of 15,000 cells per cm² and incubated for 20 min at 37° C. Fusion was stopped by exchange of the fusion solution against the cell culture medium. 24 hours after termination of the fusion reaction, the fusion efficiency was verified by means of fluorescent microscopy and flow cytometry as well as the functional transfer of the plasmid by means of the protein expression analysis. The cells positively identified in the flow cytometry are indicated in percent. A further improvement of the functional transfer of charged molecules during the execution of the additional ultrasonic step is recognizable. The fusion itself remains nearly unchanged due to the ultrasonic treatment.

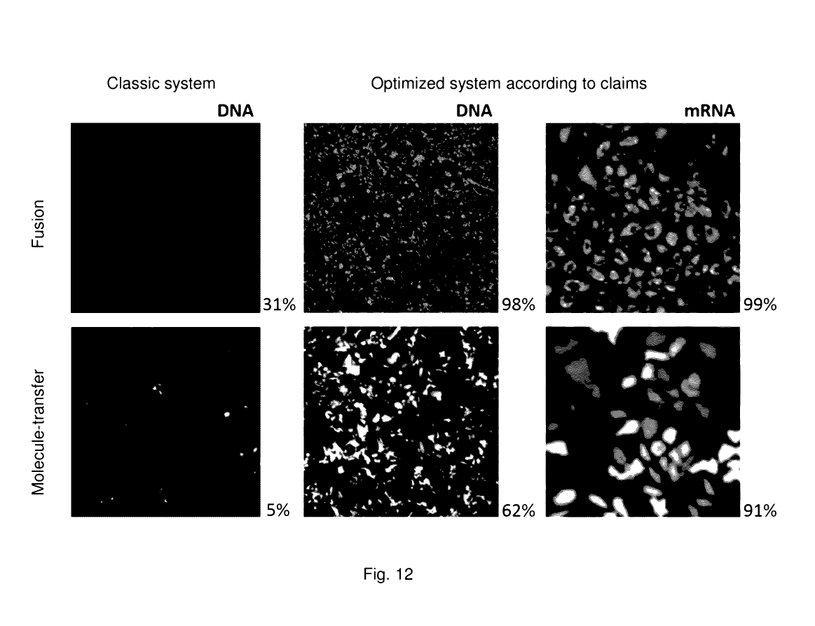

FIG. 12 illustrates conventional fusogenic liposomes and a fusion system according to the invention in direct comparison. For producing the complex of DNA and complex creator and/or RNA and complex creator (complex A), 2 μg cDNA and/or 2 μg mRNA for expression of GFP (green fluorescent protein) with polycationic substance (protamine) were respectively incubated at a weight ratio 2:1. As solution buffer, 5 μl buffer were used (10 mM Tris, pH 7.5). The incubation in the solution buffer occurred for 20 min at RT. During this time, 10 μl of a 3 mM fusogenic lipidic mixture were suspended in the ultrasonic bath at 36 kHz and 200 W for 10 min at RT in order to obtain fusogenic liposomes of middle size of approximately 340 nm.

The lipid composition of the fusogenic liposomes consisted of positively charged lipid, fusogenic molecule and neutral lipid at a weight ratio 1:0.1:1 in 20 mM HEPES, pH 7.4. After termination of the incubation times, 1 μl of a 100 mM NaCl and 0.5 μM Triton X-100 solution were added to the fusogenic liposomes and incubated with finished complex A for 20 min at 36 kHz and RT in the ultrasonic bath. 10 μl of the arising fusogenic complex A liposomes were diluted with PBS at a ratio 1:50 and again treated for 5 min at the same parameters in the ultrasonic bath.

The diluted complex A liposomes thereafter were added instead of the cell culture medium to adherent CHO (Chinese hamster ovary) cells, which had been seeded before in a density of 15,000 cells per cm² and incubated for 20 min at 37° C. Fusion was stopped by exchange of the fusion solution against the cell culture medium.

In comparison thereto, 2 μg of the identical DNA without complexation as well as without further modification (no triton, no cation, no additional ultrasonic step) with fusogenic liposomes of same concentration and composition were incubated and subsequently, cells for fusion with same treatment were provided (left). 24 hours after termination of the fusion reaction for DNA transfer as well as 3 hours for mRNA transfer, the fusion efficiency was verified by means of fluorescent microscopy and flow cytometry as well as the functional transfer of the plasmid by means of protein expression analysis. The cells positively identified in the flow cytometry are indicated in percent.

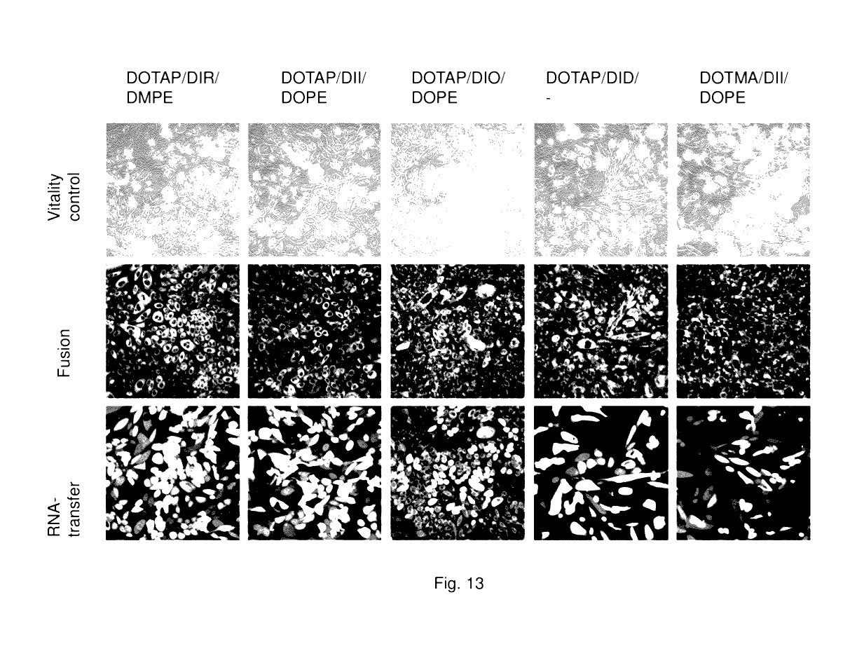

In FIG. 13, the use of different substances from the groups A, B, and C for producing a fusion system according to the invention is shown. For producing the complex of RNA and protamine as complex creator, 2 μg mRNA for expression of GFP (green fluorescent protein) were incubated with protamine at a weight ratio 2:1. As solution buffer, 5 μl buffer were used (10 mM Tris, pH 7.5). The incubation in the solution buffer occurred for 20 min. at RT. During this time, 10 μl of a 3 mM fusogenic lipidic mixture were suspended in the ultrasonic bath at 36 kHz and 200 W for 10 min at RT in order to obtain liposomes of middle size of approximately 340 nm.

As components A, B, and C of the lipid composition of the fusogenic liposomes, the substances indicated in the Figure were used. The mixture ratio of the components A:B:C was always set up similarly with a weight ratio of 1:0.1:1. Only for the preparation with the lacking component C (DOTAP/DiD/−), had the ratio 1:0.2. Mixtures were prepared in 20 mM HEPES, pH 7.4. After termination of the incubation times, 1 μl of a 100 mM NaCl and 0.5 μM triton X-100 solution were added to the fusogenic liposomes and incubated with finished complex A for 20 min at 36 kHz and RT in the ultrasonic bath. 10 μl of the arising fusogenic complex A liposomes were diluted with PBS at a ratio 1:50 and again treated for 5 min at the same parameters in the ultrasonic bath.

The diluted complex A liposomes were in the following added instead of the cell culture medium to adherent CHO (Chinese hamster ovary) cells, which had been seeded before in a density of 15,000 cells per cm² and incubated for 20 min at 37° C. Fusion was stopped by exchange of the fusion solution against the cell culture medium.

3 hours after termination of the fusion reaction, the fusion efficiency was verified by means of fluorescent microscopy as well as the functional transfer of mRNA by means of protein expression analysis. All compositions showed a very high fusion efficiency and a good transfer of charged molecules.

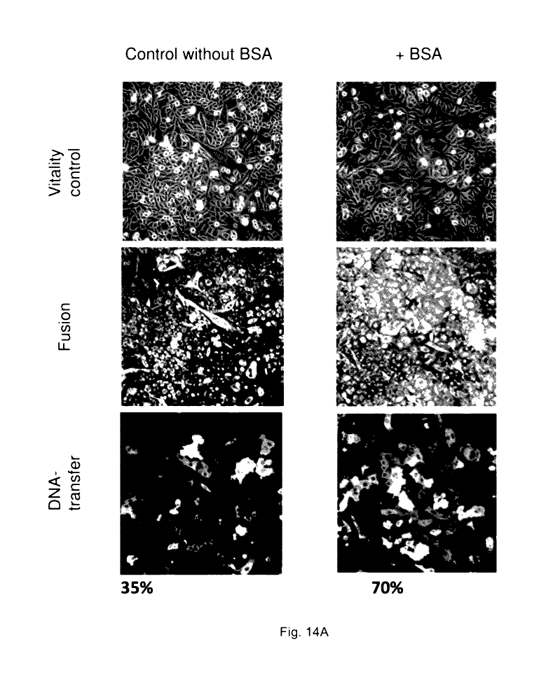

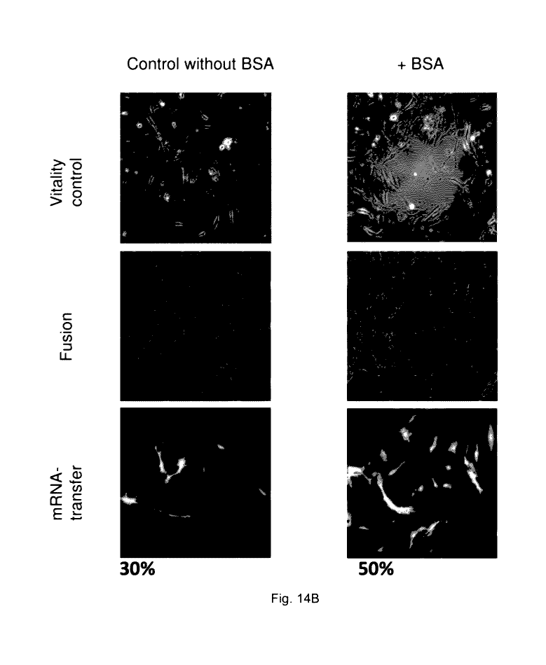

FIG. 14A illustrates the influence of albumins to the transfer efficiency of charged molecules by fusogenic liposomes. For producing the complex of DNA and complex creator (complex A), 2 μg cDNA for expression of GFP (green fluorescent protein) with polycationic substance (protamine) were incubated at a weight ratio of 2:1. As solution buffer, 5 μl buffer were used (10 mM Tris, pH 7.5). 1 μl of a 0.5 mM solution of BSA (Bovines Serum Albumin) was added to this buffer. The incubation in the solution buffer occurred for 5 min at RT. During this time, 10 μl of a 3 mM fusogenic lipid mixture were suspended in the ultrasonic bath at 46 kHz and 50 W for 10 min at RT in order to obtain fusogenic liposomes of middle size of approximately 340 nm.

The lipid composition of the fusogenic liposomes consisted of positively charged lipid (DOTAP), fusogenic molecule (DiR) and neutral lipid (DOPE) at a weight ratio 1:0.1:1 in 20 mM HEPES, pH 7.4. After termination of the incubation times, 1 μl of a 1 mM NaCl and 0.5 μM triton X-100 solution were added to the fusogenic liposomes and incubated with finished complex A for 10 min at 46 kHz (50 W) and RT in the ultrasonic bath. 10 μl of the arising fusogenic complex A liposomes were diluted with PBS at a ratio of 1:50 and again treated for 5 min at the same parameters in the ultrasonic bath.

The diluted complex A liposomes in the following were added instead of the cell culture medium to adherent CHO (Chinese hamster ovary) cells, which had been seeded before in a density of 15,000 cells per cm2 and incubated for 20 min at 37° C. Fusion was stopped by exchange of the fusion solution against the cell culture medium. 24 hours after termination of the fusion reaction, the fusion efficiency was verified by means of fluorescent microscopy and flow cytometry as well as the functional transfer of the plasmid by means of protein expression analysis. The cells positively identified in the flow cytometry are indicated in percent.

FIG. 14B illustrates the influence of albumins to the transfer efficiency of charged molecules by fusogenic liposomes. For producing the complex of mRNA and complex creator (complex A), 2 μg mRNA for expression of GFP (green fluorescent protein) with polycationic substance (protamine) were incubated at a weight ratio of 2:1. As solution buffer, 5 μl buffer were used (10 mM Tris, pH 7.5). 3 μl of a 0.5 mM solution of HAS (Human Serum Albumin) was added to this solution. The incubation in the solution buffer occurred for 5 min at RT. During this time, 10 μl of a 3 mM fusogenic lipid mixture were suspended in the ultrasonic bath at 46 kHz and 50 W for 10 min at RT in order to obtain fusogenic liposomes of middle size of approximately 340 nm.

The lipid composition of the fusogenic liposomes consisted of positively charged lipid (DOTAP), fusogenic molecule (DiR), and neutral lipid (DOPE) at a weight ratio of 1:0.1:1 in 20 mM HEPES, pH 7.4. After termination of the incubation times, 1 μl of a 1 mM NaCl and 0.5 μM of a triton X-100 solution were added to the fusogenic liposomes and incubated with finished complex A for 10 min at 46 kHz (50 W) and RG in the ultrasonic bath. 10 μl of the arising fusogenic complex A liposomes were diluted with PBS at a ratio 1:50 and again treated for 5 min at the same parameters in the ultrasonic bath.

The diluted complex A liposomes were in the following added instead of the cell culture medium to primary myofibroblasts of the mouse, which are difficult to fuse, differentiated, and which had been seeded before in a density of 15,000 cells per cm², and are incubated for 20 min at 37° C. Fusion was stopped by exchange of the fusion solution against cell culture medium. 3 hours after the termination of the fusion reaction, the fusion efficiency was verified by means of fluorescent microscopy and flow cytometry as well as the functional transfer of the plasmid by means of protein expression analysis. The cells positively identified in the flow cytometry are indicated in percent.

From the plurality of different embodiments it results that the method according to the invention reveals a fusion mixture, which reliably allows a transfer by means of fusion into and/or through a lipid membrane, during which particularly the transferred molecules maintain their functionality.

1.-17. (canceled)

18. A method for producing a fusion mixture for a transfer of a charged molecule into and/or through a lipid membrane comprising:
a) providing an initial mixture comprising a positively charged amphipathic molecule A, an aromatic molecule B with hydrophobic range and a neutral, amphipathic molecule C, whereby the molecule types are at hand in a ratio A:B:C of 1-2:0.02-1:0-1 mol/mol,
b) generating a fusogenic liposome by absorption of the initial mixture in a watery solvent,
c) providing a charged molecule,
d) forming a complex from the charged molecule and a neutralizing agent, and
e) incubating the complex with the fusogenic liposome so that a fusion mixture is obtained.

19. The method according to claim 18, wherein step d) occurs in a manner that the complex has a zeta potential of −50 mV to 0 mV.

20. The method according to claim 18, wherein before step e) an adding of cations occurs in order to stabilize the complex.

21. The method according to claim 20, wherein the cations are added with a concentration of 0 to 1 mM.

22. The method according to claim 18, wherein step d) comprises an adding of albumin.

23. The method according to claim 18, wherein before, during and/or after step e) a lipid membrane destabilizing agent is added.

24. The method according to claim 23, wherein the lipid membrane destabilizing agent is a detergent with a head-to-chain aspect ratio of at least 1:1.

25. The method according to claim 18, wherein step b) and/or step e) comprise an implementation of an ultrasonic treatment and/or an implementation of a high-pressure homogenization.

26. The method according to claim 18, wherein after step e), a dilution with a buffer with an osmolarity of 200 mOsm or more occurs.

27. The method according to claim 26, wherein the buffer has a pH-value of 5 to 10.

28. The method according to claim 18, wherein during the dilution and/or after the dilution, an ultrasonic treatment and/or a pressurization is carried out.

29. The method according to claim 18, wherein molecule A and/or molecule C are a lipid or a lipid analogon.

30. The method according to claim 18, wherein molecule A and/or molecule C, at contact with water generate a planar or nearly planer bilayer.

31. A fusion mixture produced by the method set forth in claim 18.

32. A method for fusion of a lipid membrane comprising:
using a fusion mixture produced by the method set forth in claim 18;
wherein the lipid membrane comprises a cell membrane or a compound of a cell membrane.

33. A method for transfer of a charged molecule in and/or through a lipid membrane by means of fusion, comprising:
implementation of the method according to claim 18, and bringing into contact the fusion mixture with a lipid membrane so that the fusion mixture fuses with the lipid membrane.

34. The method according to claim 33, wherein the lipid membrane is a cell membrane or a compound of a cell membrane.
